(19) United States
(12) Patent Application Publication
(10) Pub. No.: US 2024/0181062 A1
(43) Pub. Date: Jun. 6, 2024

(54) RED-SHIFTED PHOTOLIPIDS AND NANOPARTICLES FORMED FROM SAME

(71) Applicants: New York University, New York, NY (US); The University of British Columbia, Vancouver (CA); Ludwig-Maximilians-Universität München, München (DE)

(72) Inventors: Dirk TRAUNER, New York, NY (US); Johannes MORSTEIN, Bremen (DE); Pieter Rutter CULLIS, Vancouver (CA); Dominik WITZIGMANN, Vancouver (CA); Nisha CHANDER, Vancouver (CA); David B. KONRAD, München (DE)

(21) Appl. No.: 18/551,390

(22) PCT Filed: Mar. 21, 2022
(86) PCT No.: PCT/US2022/021150

§ 371 Date: Sep. 19, 2023

Related U.S. Application Data
(60) Provisional application No. 63/163,693, filed on Mar. 19, 2021.

(51) Int. Cl. A61K 41/00; A61K 9/127; A61K 31/704; C07F 9/10

(52) U.S. Cl. CPC A61K 41/0042; A61K 9/1271; A61K 31/704; C07F 9/106

(57) ABSTRACT

Provided are photoswitchable glycerophospholipids having a photoswitchable group having the following structure: or The photoswitchable glycerophospholipids can be isomerized by red spectral light. Also disclosed are nanovesicles (e.g., liposomes). Also disclosed are methods of making the photoswitchable glycerophospholipids and nanovesicles formed therefrom. The present disclosure further provides methods of using the nanovesicles. e.g., for targeted delivery of cargo. A photoswitchable glycerophospholipid may have the following structure: Structure I.

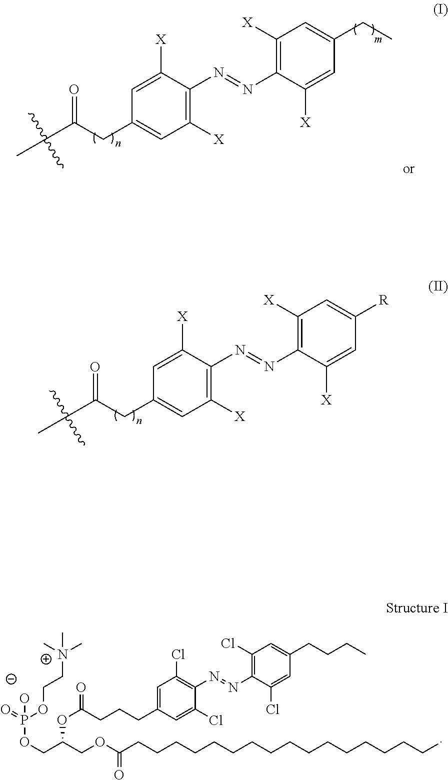

### CROSS REFERENCE TO RELATED APPLICATIONS

This application claims priority to U.S. Provisional Patent Application No. 63/163,693, filed on Mar. 19, 2021, the disclosure of which is incorporated herein by reference.

### BACKGROUND OF THE DISCLOSURE

Photoswitchable azobenzene phospholipid molecules (short ‘photolipids’) that are embedded in a phospholipid bilayer are an intriguing tool to control membrane properties with light. The bottleneck for photolipid applications has been set by the limited spectral range at which the switching process is efficient.

Lipid nanoparticles (LNP) are the leading drug delivery platform in the clinic for systemic applications. Unilamellar LNPs with diameters less than 100 nm are favoured for delivery of small molecular drugs. Solid core systems are better suited for delivery of macromolecular genetic drugs, such as siRNA or mRNA. More than 10 LNP therapeutics have been approved by the US FDA and other regulatory agencies. Most of these are liposomes containing anticancer drugs that exhibit reduced toxicity and enhanced efficacy compared to the free drug.

Robust techniques exist for achieving efficient drug encapsulation in <100 nm diameter liposomal systems that exhibit long half-lives in the circulation (up to 24 h in humans) and preferential accumulation at tumor sites following intravenous injection. However, a major limitation is that they do not selectively leak their contents after arrival at the target site. This severely limits the improvement in therapeutic index that can be gained by liposomal delivery. Technologies that trigger release of liposomal contents either at or near the target site would have significant benefits. This is particularly true given that liposomes containing cancer drugs can exhibit maximum tolerated doses (MTD) that are up to five times higher than those of free drug and thus are systemically much less toxic.

There have been many attempts to develop triggered release systems for liposomal systems containing anticancer drugs. To this end, thermosensitive lipids or metallic nanoparticles (such as or gold nanoparticles or iron oxide nanoparticles) tethered to the liposome have been employed. These systems give rise to liposomes that leak contents in response to local heating or irradiation. Many systems, however, are quite complex, limiting their manufacturing scalability or require the development of a specific device to trigger release. In addition, several reported systems exhibit poor drug loading/retention and relatively short circulation lifetimes resulting in off-target release and reduced ability to access the desired target tissues. As a result, only one triggered release technology has progressed to late stage clinical trials to date: ThermoDox—a liposomal doxorubicin formulation for the treatment of inoperable hepatocellular carcinoma (HCC), where drug release is stimulated by a mild hyper-thermic trigger. The lipid composition of ThermoDox is significantly different as compared to the approved Doxil formulation to ensure a relatively sharp transition temperature. However, this resulted in a formulation with different pharmacokinetics and a relatively short circulation lifetime. Despite more than 30 years of efforts, the only triggered release system (in response to local heating) that had made it into the clinic failed in phase III.

Alternative attempts to develop approaches for light-triggered drug release from liposomal targets have not progressed to a clinical setting due to inefficient drug encapsulation and release, lack of straightforward and scalable methods of manufacture, and difficulty in selecting the clinical entry point. In addition, there is a range of competitive technologies including simple and effective device-only ablative methods such as microwave and radiofrequency ablation, as well as surgery and radiotherapy. The implementation of light-triggered drug release systems, however, could benefit from approved phototherapies and photodynamic therapies (PDT) as well as emerging technologies to deliver light deep within patients.

Encapsulation of small molecule drugs in long-circulating LNPs can reduce toxic side effects and enhance accumulation at tumor sites. A fundamental problem, however, is the slow release of encapsulated drugs from these liposomal systems at the disease site resulting in limited therapeutic benefit. Ideally, new systems for light-triggered release should closely mimic the composition and properties of clinically approved LNPs in terms of composition, size, loading, and stability. The approved systems Doxil, Ambisome and Marqibo all use saturated lipids that contain choline headgroups at approximately equimolar levels with cholesterol as the primary lipid constituents. Such phosphatidylcholine lipid-cholesterol compositions can be readily formulated into liposomal systems with diameters <100 nm that can be efficiently loaded with weakly basic drugs, such as doxorubicin, and display long circulation lifetimes following i.v. administration. With the constantly evolving field of photo pharmacology, approaches to include photosensitizers like porphyrin into stealth liposomes have helped advance the light-triggered drug release concept to have more clinical translatability. These types of systems take advantage of the well-established field of PDT.

### SUMMARY OF THE PRESENT DISCLOSURE

The present disclosure provides photoswitchable glycerophospholipids. The photoswitchable glycerophospholipids can be isomerized by red spectral light. Also disclosed are nanovesicles (e.g., liposomes) comprising the photoswitchable glycerophospholipids. Also disclosed are methods of making the photoswitchable glycerophospholipids and nanovesicles formed therefrom. The present disclosure further provides methods of using the nanovesicles, e.g., for targeted delivery of cargo.

In an aspect, provided are photoswitchable glycerophospholipids. The photoswitchable glycerophospholipids comprise a head group comprising a phosphate group; one or more C₈-C₂₄ acyl groups; and one or more photoswitchable groups. The photoswitchable groups are connected to the glycerol backbone, the alkyl chains of the acyl groups or both. The photoswitchable group isomerizes upon exposure to electromagnetic radiation in the visible spectrum. The photoswitchable groups may isomerize to the cis confirmation when exposed to red electromagnetic radiation.

In an aspect, the present disclosure provides nanovesicles (e.g., liposomes). The nanovesicles comprise one or more photoswitchable glycerophospholipids of the present disclosure, one or more glycerophospholipid that are not attached to a photoswitchable group, and one or more sterols (e.g., cholesterol). The nanovesicles can further comprise one or more cargo molecules. The nanovesicle can be exposed with electromagnetic radiation such that cargo molecules are released from the nanoparticle.

In an aspect, the present disclosure provides a composition. The composition comprises a nanovesicle of the present disclosure.

In an aspect, the present disclosure provides methods of preparing nanovesicles (e.g., liposomes). The methods also include methods of loading the nanovesicles with one or more cargo molecules.

In an aspect, the present disclosure provides methods of using a nanovesicle. The nanovesicles may be used to deliver cargo molecules to a target site of an individual.

### BRIEF DESCRIPTION OF THE FIGURES

For a fuller understanding of the nature and objects of the disclosure, reference should be made to the following detailed description taken in conjunction with the accompanying figures.

FIG. 1 shows synthesis of red azo-PC.

FIG. 2 shows (A) ¹H NMR data, (B) ¹³C NMR data, and (C) ³¹P NMR data for (R)-2-((4-(4-((4-butyl-2,6-dichlorophenyl)diazenyl)-3,5-dichlorophenyl)butanoyl)oxy)-3-(stearoyloxy)propyl (2-(trimethylammonio)ethyl) phosphate (red-azo-PC).

FIG. 3 shows design of a light-triggered drug release system. A. Chemical structure of DSPC and its photoswitchable analog AzoPC. The azobenzene can be isomerized from the trans to the cis form at 365 nm. B. Schematic for light induced drug release from photoactivatable LNPs (paLNPs). Low levels of trans AzoPC are incorporated in a DSPC-cholesterol liposomal system loaded with doxorubicin. Photoisomerization of AzoPC induces drug release.

FIG. 4 shows substitution of up to 10 mol % trans AzoPC for DSPC in DSPC-cholesterol liposomes allows for efficient loading of doxorubicin (DOX). (A) AzoPC photoswitching kinetics in paLNPs vs. free dissolved AzoPC. Samples at 3 mg/mL AzoPC concentration were used for absorbance measurements. (B) Doxorubicin entrapment efficiency of paLNP formulations with increasing amounts of AzoPC. (C) Schematic for remote loading of doxorubicin into paLNPs incorporating AzoPC in the trans form. paLNPs containing 300 mM ammonium sulphate in the aqueous core and suspended in PBS were mixed with doxorubicin at a drug to lipid (wt/wt) ratio of 0.1. The mixture was heated in a water bath at 65° C. for 30 min, following removal of unentrapped doxorubicin via dialysis. (D) Schematic for remote loading of doxorubicin into paLNPs with AzoPC in the cis form. paLNPs were subject to UV irradiating at 365 nm for 5 min, following the same drug loading procedure as above. € Comparison of doxorubicin loading efficiency in paLNP formulations containing 10% AzoPC before and after photoswitching via irradiation with UV-A light (365 nm). Error bars represent SEM **p<0.01, n.s., not significant, Student's t-test.

FIG. 5 shows presence of serum proteins significantly inhibited drug release from paLNP compared to paLNP in PBS. (A) paLNP particles containing a substitution of 10-30 mol % trans AzoPC for DSPC in control DSPC-cholesterol liposomes suspended in PBS were irradiated with a UV-A light source (365 nm) for 5 minutes followed by storage in the dark at room temperature for 1 hr. Samples were assayed for drug release (doxorubicin, DOX) by measuring absorbance at 492 nm. (B) paLNP particles containing 10 mol % AzoPC suspended in PBS or DMEM media containing 10% FBS were irradiated with a UV-A light source (365 nm) for 5 minutes followed by storage in the dark at room temperature for 24 hours. Samples were assayed for drug release (Dox) by measuring absorbance at 492 nm at 0 h, 1 h, 4 h, 8 h and 24 h timepoints. Error bars represent SEM *p<0.1, Student's t-test.

FIG. 6 shows pulsed irradiation (365 nm) of paLNP (10 mol % AzoPC) results in triggered release of doxorubicin both in the absence and presence of serum. (A) ‘Cell-DISCO’ setup for pulsed irradiation using a single-board microcontroller (e.g. Arduino), power relay module board, LED plate, and power supplies. (B) Irradiation protocol for pulsed LED starting with a 5 min initial irradiation followed by 75 ms irradiation pulses. (C) Light-triggered doxorubicin (DOX) release from Control-LNP and paLNP (10 mol % AzoPC) using pulsed LED irradiation (365 nm) for 24 h at 20° C. in PBS or (D) DMEM media containing 10% FBS. Error bars represent SEM **p<0.01, ***p<0.001, ****p<0.0001, n.s., not significant, Student's t-test.

FIG. 7 shows doxorubicin loaded paLNP show evidence of drug release following pulsed irradiation (365 nm) as detected employing cryo-TEM. (A) Representative cryo-TEM images of control DSPC-cholesterol liposomes and paLNP containing 10 mol % AzoPC prior to UV irradiation and post UV irradiation using a pulsed LED at 365 nm over a 24 h period. (B) Comparison of thickness of doxorubicin (DOX) crystal within the liposomes pre and post UV irradiation. Error bars represent SEM *p<0.1, n.s., not significant, Student's t-test.

FIG. 8 shows doxorubicin (DOX) released from drug loaded paLNP following irradiation is biologically active. (A) Influence of irradiation on the viability of HuH7 cells incubated with increasing concentrations of doxorubicin in free form or encapsulated in Control-LNP or paLNP. Cells were incubated in the presence of 0, 0.1, 1, 10 and 100 μM DOX concentrations and were irradiated for 5 min at 365 nm and then subjected to pulsed irradiation at 365 nm for 24 h. Drug release was reflected by decreased cell viability. (B) Confocal images of HuH7 cells treated with Control-LNP and paLNP with and without UV irradiation. Samples with 10 μM DOX concentrations were irradiated for 5 min at 365 nm following, after which samples were subjected to pulsed irradiation at 365 nm for 24 h.

FIG. 9 shows pulsed deep-red light irradiation (660 nm) of red-paLNP (10 mol % redAzoPC) results in triggered release of doxorubicin. (A) Chemical structure of photoswitchable analog redAzoPC. The tetra-ortho-chloro-azobenzene can be isomerized from the trans to the cis form at 660 nm. (B) Light-triggered doxorubicin (DOX) release from Control-LNP and red-paLNP (10 mol % redAzoPC) using pulsed LED irradiation (660 nm) for 24 h at 20° C. in PBS or (C) DMEM media containing 10% FBS. Error bars represent SEM **p<0.01, ***p<0.001, n.s., not significant, Student's t-test. (D) Influence of irradiation on the viability of HuH7 cells incubated with increasing concentrations of doxorubicin in its free form or encapsulated in Control-LNP or red-paLNP. Cells were incubated in the presence of 0, 0.1, 1, 10 and 100 μM DOX concentrations and were irradiated for 5 min at 660 nm and then subjected to pulsed irradiation at 660 nm for 24 h. Doxorubicin release was reflected by decreased cell viability.

FIG. 10 shows assessment of LNP pharmacokinetics in vivo in transgenic zebrafish. Control LNP and paLNPs were injected intravenously into transgenic Tg(kdrl:EGFP) zebrafish expressing enhanced green fluorescent protein in their vasculature. LNPs were fluorescently labeled with DiD. (A) Confocal images of tail region were acquired at 2 h and 24 h post injection (hpi). (B) Systemic circulation properties were quantified based on fluorescence signals in the dorsal aorta (box 1). (C) LNP extravasation into tissue was quantified between the intersegmental vessels (box 2). Error bars represent SEM.

FIG. 11 shows doxorubicin release in zebrafish embryos in presence or absence of pulsed light trigger. Control LNP and paLNPs were injected intravenously into wildtype zebrafish embryos 48 h post fertilization (hpf) (2 nL, 3 mg/ml doxorubicin). After 24 h, zebrafish were exposed to pulsed irradiation and confocal images of tail region were acquired at 48 h post injection (hpi). (A) and (B) Representative images of doxorubicin release (white signal) from Control LNP, (A) paLNP, or (B) red-paLNP in presence or absence of pulsed UV-A (365 nm) or deep-red light (660 nm) irradiation. White arrows indicate exemplified areas with enhanced doxorubicin release. (C) Quantitative image analysis of doxorubicin release. Errors bars represent SEM. *p<0.1, **p<0.01.

FIG. 12 shows influence of irradiation on the viability of HuH7 cells incubated with increasing concentrations of doxorubicin in free form or encapsulated in control or paLNP. Cells were incubated in the presence of 0, 0.1, 1, 10 and 100 μM Dox concentrations. At 6 h post transfection, cells were irradiated for 5 min at 365 nm and stored at 37° C. for 24 h. Drug release was demonstrated by cell viability.

FIG. 13 shows confocal images of HuH7 cells treated with control-LNP and paLNP with and without UV irradiation. Samples with 1 μM dox concentrations were irradiated for 5 min at 365 nm following which samples were subjected to pulsed irradiation at 365 nm for 24 h.

FIG. 14 shows control LNP, paLNP and red-paLNPs fluorescently labeled with DiD were injected intravenously into wild-type zebrafish for assessment of pharmacokinetic properties. A. Confocal images of tail region were acquired at 24 h post injection (hpi) for control LNP and paLNP with and without pulsed irradiation at 365 nm for 24 h. B. Confocal images of tail region were acquired at 24 h post injection (hpi) for control LNP and red-paLNP with and without pulsed irradiation at 660 nm for 24 h.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

The present disclosure provides photoswitchable glycerophospholipids. The photoswitchable glycerophospholipids can be isomerized by red spectral light. Also disclosed are nanovesicles (e.g., liposomes) comprising the photoswitchable glycerophospholipids. Also disclosed are methods of making the photoswitchable glycerophospholipids and nanovesicles formed therefrom. The present disclosure further provides methods of using the nanovesicles, e.g., for targeted delivery of cargo.

In an aspect, provided are photoswitchable glycerophospholipids. The photoswitchable glycerophospholipids comprise a head group comprising a phosphate group; one or more C₈-C₂₄ acyl groups (e.g., C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄); and one or more photoswitchable groups. The photoswitchable groups are connected to the glycerol backbone, the alkyl chains of the acyl groups or both. The photoswitchable group isomerizes upon exposure to electromagnetic radiation in the visible spectrum (e.g., 350-700 nm, including every nm value and range therebetween (e.g., 365-660 nm)). The photoswitchable groups may isomerize to the cis confirmation when exposed to red electromagnetic radiation.

In various examples, a phospholipid is a phospholipid other than a glycerophospholipid. For example, rather than have an ester linkage, the phospholipid has an amide, ether, or alkyl linkage.

The photoswitchable glycerophospholipids may comprise various photoswitchable groups. The photoswitchable groups may be at any position on the alkyl chain of the acyl group, or on either or both alkyl chains in the event there are two acyl groups (e.g., the photoswitchable group may be a substituent of the acyl group or be part of the longest linear chain of the acyl group). The photoswitchable groups are azobenzene groups, which are tetra-ortho-substituted (the four positions ortho to the azo group of the azobenzene). For example, the photoswitchable group has the following structure:

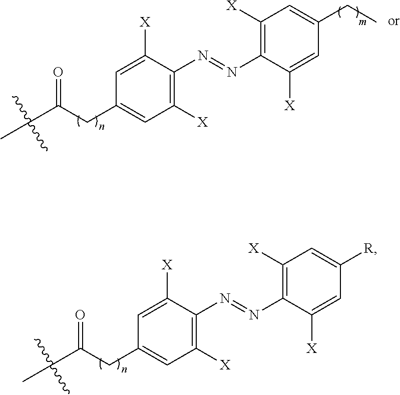

where each X is individually chosen from —F, —Cl, -OMe, and combinations thereof; R is —H, —NO₂, amine groups (e.g., second amines or tertiary amines), ether groups (e.g., -OMe, -OEt, and the like), —Br, or —I; n is 0-12 (e.g., 0, 1, 2, 3, 4, 5, 6, 7, 8, 9, 10, 11, or 12); and m is 0-12 (e.g., 0, 1, 2, 3, 4, 5, 6, 7, 8, 9, 10, 11, or 12). Various combinations of X groups may be used. For example, all the X groups are chloro groups, all the X groups are fluoro groups, all the X groups are methoxy groups, or the X groups are a combination of chloro groups, fluoro groups, and methoxy groups. Without intending to be bound by any particular theory, it is considered that the substitution at the ortho positions red shift the isomerization of the azobenzene group. Typically, azobenzene groups isomerize in the UV and/or blue spectral range. The photoswitchable groups of the present disclosure isomerize in the spectral range of 350 to 700 nm, including all integer nm values and ranges therebetween (e.g., 365-660 nm).

In various examples, the photoswitchable groups of the present disclosure isomerize to the cis confirmation from the trans confirmation when exposed to electromagnetic radiation at 660, 550, or 365 nm and from the cis confirmation to the trans confirmation when exposed to electromagnetic radiation at 395 nm or 465 nm.

For example, the photoswitchable group has the following structure:

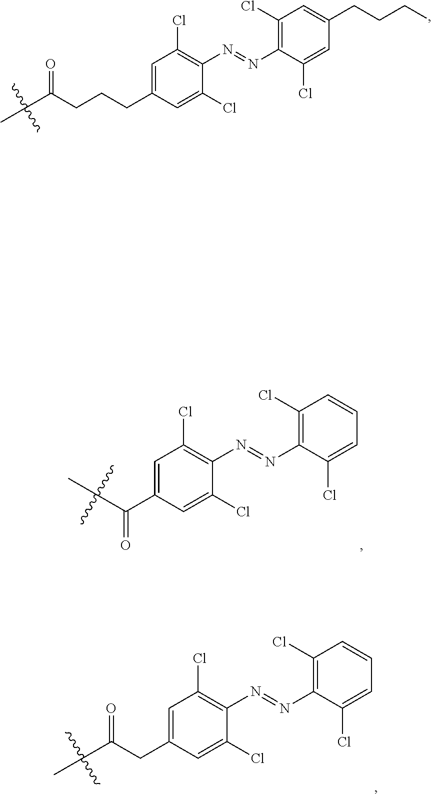

or a combination thereof. In various examples, the photoswitchable group does not have the following structure:

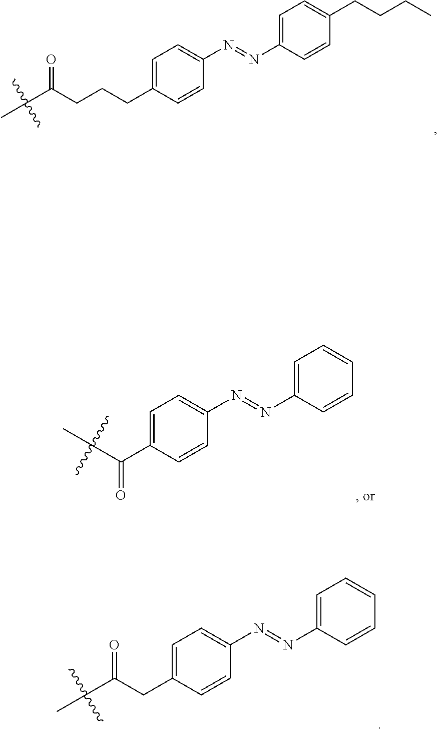 [SMILES: *C(=O)CCCc1ccc(/N=N/c2ccc(CCCC)cc2)cc1 ; *C(=O)c1ccc(/N=N/c2ccccc2)cc1 ; *C(=O)Cc1ccc(/N=N/c2ccccc2)cc1]

Various head groups may be used. Various head groups of glycerophospholipids are known in the art. For example, the head group is chosen from an ethanolamine group, a choline group, a serine group, a glycerol group, a myo-inositol 4,5-bisphosphate group, a phosphatidyl glycerol group, and the like.

A non-limiting example of a photoswitchable glycerophospholipid includes:

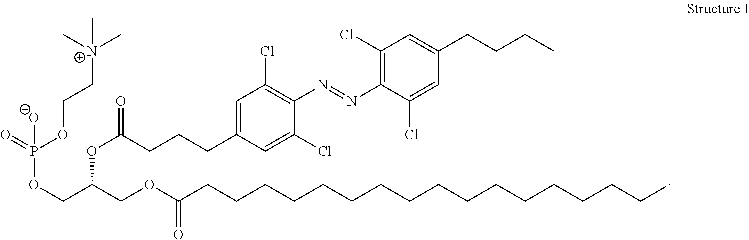

In an aspect, the present disclosure provides nanovesicles (e.g., liposomes). The nanovesicles comprise one or more photoswitchable glycerophospholipids of the present disclosure, one or more glycerophospholipid that are not attached to a photoswitchable group, and one or more sterols (e.g., cholesterol). The nanovesicles can further comprise one or more cargo molecules. The nanovesicle can be exposed with electromagnetic radiation such that cargo molecules are released from the nanoparticle.

The nanovesicles may be unilamellar nanovesicles. In various examples, the nanovesicles are unilamellar or multilamellar. The nanovesicles may have a longest linear dimension (e.g., diameter) of 10-150 nm, including every 0.1 nm value and range therebetween (e.g., ˜56-58 nm). In various examples, the nanoparticles have a monodisperse population. For example, the PDI is less than 1, less than 0.5, or less than 0.1.

Various photoswitchable glycerophospholipids may be used. Various examples of photoswitchable glycerophospholipids are provided herein. For example, the photoswitchable glycerophospholipids may have any one of the following head groups: an ethanolamine group, a choline group, a serine group, a glycerol group, a myo-inositol 4,5-bisphosphate group, a phosphatidyl glycerol group, or the like. Various photoswitchable groups are described herein. Various concentrations of the photoswitchable glycerophospholipids may be used. For example, the concentration of photoswitchable glycerophospholipid is 0.1-80 mol %, including every 0.01 mol % value and range therebetween (e.g., 10 mol %). For example, a nanovesicle comprises a photoswitchable glycerophospholipid may be Structure I.

Various glycerophospholipids may be used. Various examples glycerophospholipids are known in the art. Non-limiting examples of glycerophospholipids include phosphatidyl choline, phosphatidyl ethanolamine, phosphatidyl inositol, phosphatidyl serine, bisphosphatidyl glycerol, lyso derivatives thereof, and the like, and combinations thereof. Various concentrations of glycerophospholipids may be used. For example, the concentration of the glycerophospholipid is 5-99.9 mol %, including every 0.01 mol % value and range therebetween.

Various sterols may be used for the nanovesicles. The sterols may be used to aid in stabilization of the nanovesicle. Examples of sterols are known in the art. The sterols may be animal sterols or plant sterols. Examples of sterols include cholesterol, sitosterol, stigmasterol, cholestanol, and the like, and combinations thereof. Various concentrations of sterol may be used. For example, the concentration of the sterol is 1 to 70 mol %, including every 0.01 mol % value and range therebetween.

The nanovesicles may carry various cargo molecules. The cargo molecules may be incorporated in in the bilayer of the nanovesicle or may be present in the aqueous compartment or may have some portion of the cargo molecule in the bilayer and some portion of the cargo molecule in the aqueous compartment. Some portion of the cargo molecule may exposed to the outside of the nanovesicle.

A wide variety of cargo may be loaded into the nanovesicles of the present disclosure and delivered to desired locations using electromagnetic radiation in the visible spectrum. For example, bioactive or therapeutic agents, diagnostics agents, targeting agents, pharmaceutical substances, and/or drugs can be encapsulated within the interior of the nanovesicles. This includes water soluble drugs and also drugs that are weak acids or bases that can be loaded via chemical gradients and concentrated in the aqueous core of the nanovesicle. Thus, in various embodiments, the nanovesicle comprises an active agent encapsulated therein, such as a therapeutic agent or a diagnostic agent.

The cargo molecules may be pharmaceutical drugs (e.g., the pharmaceutical drug has a pKₐ of greater than 7 (e.g., is a weak base)). For example, the cargo molecule is chosen from doxorubicin, nucleic acids (e.g., siRNA, mRNA, plasmids, and the like, and combinations thereof), peptides, and the like, and combinations thereof. Additionally the cargo molecules may be hydrophobic, hydrophilic, or amphiphilic.

The term “therapeutic agent” refers to any chemical moiety that is a biologically, physiologically, or pharmacologically active substance. Examples of therapeutic agents, also referred to as “drugs,” are described in well-known literature references such as the Merck Index, the Physician's Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject. Drugs that are known be loaded via active gradients include doxorubicin, irinotecan, gemcitabine, epirubicin, topotecan, vincristine, mitoxantrone, ciprofloxacin, cisplatin and daunorubicin. These drugs can be loaded in and released from the nanovesicles. Therapeutic cargo also includes various antibiotics (such as gentamicin) or other agents effective against infections caused by bacteria, fungi, parasites, or other organisms, anti-inflammatory agents, or antiviral agents.

A “diagnostic” or “diagnostic agent” is any chemical moiety that may be used for diagnosis. For example, diagnostic agents include imaging agents, such as those containing radioisotopes such as indium or technetium; contrast agents containing iodine or gadolinium chelates.

The nanovesicles of the present disclosure may have various desirable features. The nanovesicles may be unilamellar. Upon exposure to electromagnetic radiation, the photoswitchable glycerophospholipid of the nanovesicle isomerize, which results in the release of the cargo molecules encapsulated in the nanovesicle. Additionally, in the presence of serum, the nanovesicles of the present disclosure comprising a tetra-ortho-chloro-azobenzene-modified AzoPC release more drug over time the upon irradiation with red light relative to Azo-PC without chlorination at the ortho positions

In an aspect, the present disclosure provides a composition. The composition comprises a nanovesicle of the present disclosure.

A composition can comprise one or more nanovesicles in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered, as well as by the particular method used to administer the composition. Accordingly, there are a wide variety of suitable formulations of pharmaceutical compositions of the present disclosure. Additional, non-limiting examples of carriers include solutions, suspensions, emulsions, solid injectable compositions that are dissolved or suspended in a solvent before use, and the like. Injections may be prepared by dissolving, suspending, or emulsifying one or more of active ingredients in a diluent. Examples of diluents, include, but are not limited to distilled water for injection, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. Parenteral administration may be prepared and include infusions such as, for example, intramuscular, intravenous, intraarterial, intraperitoneal, subcutaneous administration, and the like. For example, composition comprises nanovesicles of the present disclosure and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as histidine, citrate, or phosphate.

In an aspect, the present disclosure provides methods of preparing nanovesicles (e.g., liposomes). The methods also include methods of loading the nanovesicles with one or more cargo molecules.

A method for preparing nanovesicles may comprise co-dissolving a sterol (e.g., cholesterol), one or more glycerophospholipids (e.g., 1,2-distearolyl-sn-glycero-3-phosphocholine (DSPC)), and one or more photoswitchable glycerophospholipids in a solvent (e.g., ethanol). The nanoparticles may then be formed using the T-tube formulation (e.g., at a total flow rate of 20 mL/min and a flow rate ratio of 3:1 aqueous:organic (v/v)). Various total lipid concentrations may be used. For example, the lipid amount is 10 μmol in 25% ethanol and 300 mM ammonium sulfate. The nanovesicles may then be dialyzed (e.g., dialyzed against 300 mM ammonium sulfate) to remove residue solvent (e.g., ethanol).

A method of loading the nanovesicle may comprise loading via a pH gradient. For example, the method comprises dialyzing the nanovesicles against a buffered solution (e.g., against phosphate buffered saline (PBS) at pH 7.4). The nanovesicles may then combined with a buffered aqueous solution (e.g., an aqueous PBS solution) comprising the one or more cargo molecules (e.g., doxorubicin). The total lipid concentration of the loaded nanovesicles may be 1.5-30 mg/mL, including every 0.1 mg/mL value and range therebetween (e.g., 3 mg/mL) and a drug:lipid (molar) ratio of 0.05:1 to 0.3 0.1, including every 0.01 ratio value and range therebetween (e.g., 0.1).

In an aspect, the present disclosure provides methods of using a nanovesicle. The nanovesicles may be used to deliver cargo molecules to a target site of an individual.

A method for delivering a cargo molecule may comprise: administering to the individual a composition comprising the nanovesicles, such that the nanovesicles are delivered to a target site; exposing the target site of the individual to red spectral electromagnetic radiation, where the cargo molecules are released from the nanovesicles and the cargo molecules are delivered to the target site of the individual. For example, the nanovesicles are irradiated with electromagnetic radiation at a wavelength necessary to induce isomerization of the photoswitchable group.

The electromagnetic radiation may be delivered by various sources. If the target area is superficial, e.g., skin, then the light can be directed to the specific area. If the target area in internal, then electromagnetic radiation can be delivered to the desired area directly by shining laser light on the target area or via fiber optic probes. In the case of a tumor, the fiber optic probe may be positioned in close proximity to the tumor or within the tumor (i.e., via a catheter or endoscopic device) to provide irradiation to a localized area. Following laser exposure, the nanovesicles may be resealed. In this manner, the opening and closing of the nanovesicles may be reversible.

Various diseases may be treated using a method of the present disclosure. For example, various cancers may be treated using a method of the present disclosure.

An individual in need of treatment may be a human or non-human mammal. Non-limiting examples of non-human mammals include cows, pigs, mice, rats, rabbits, cats, dogs, other agricultural animal, pet, service animals, and the like.

The following Statements provide various examples of the present disclosure.
Statement 1. A photoswitchable glycerophospholipid comprising a head group having a phosphate group, one or two C₈-C₂₄ acyl groups, and one or more photoswitchable groups, wherein the photoswitchable group isomerizes upon exposure to electromagnetic radiation of the visible spectrum (e.g., 350-700 nm).
Statement 2. A photoswitchable glycerophospholipid according to Statement 1, wherein the one or more photoswitchable groups have the following structure:

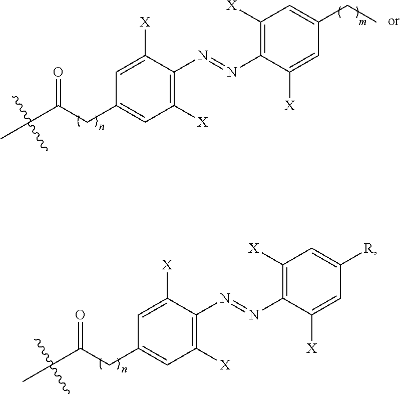

wherein each X is individually chosen from —F, —Cl, -OMe, and combinations thereof; R is —H, —NO₂, amine groups (e.g., second amines or tertiary amines), ether groups (e.g., -OMe, -OEt, and the like), —Br, or —I; n is 0-12 (e.g., 0, 1, 2, 3, 4, 5, 6, 7, 8, 9, 10, 11, or 12), and m is 0-12 (e.g., 0, 1, 2, 3, 4, 5, 6, 7, 8, 9, 10, 11, or 12).
Statement 3. A photoswitchable glycerophospholipid according to Statement 1 or Statement 2, wherein the photoswitchable group has the following structure:

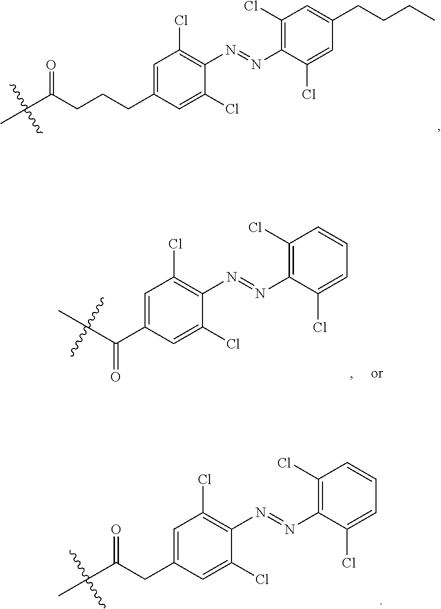 [SMILES: *C(=O)CCCc1cc(Cl)c(/N=N/c2c(Cl)cc(CCCC)cc2Cl)c(Cl)c1 ; *C(=O)c1cc(Cl)c(/N=N/c2c(Cl)cccc2Cl)c(Cl)c1 ; *C(=O)Cc1cc(Cl)c(/N=N/c2c(Cl)cccc2Cl)c(Cl)c1]

Statement 4. A photoswitchable glycerophospholipid according to any one of the preceding Statements, wherein the head group is chosen from an ethanolamine group, a choline group, a serine group, a glycerol group, a myo-inositol 4,5-bisphosphate group, and a phosphatidyl glycerol group.
Statement 5. A photoswitchable glycerophospholipid according to any one of the preceding Statements, wherein the photoswitchable glycerophospholipid has the following structure:

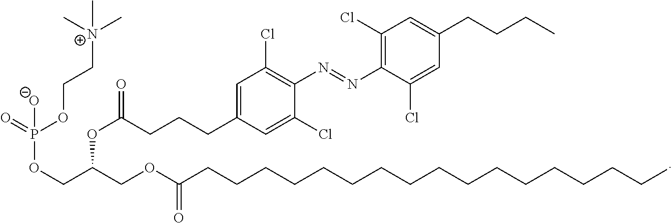

Statement 6. A nanovesicle (e.g., liposome) comprising one or more photoswitchable glycerophospholipid according to Statement 1, one or more glycerophospholipid that do not comprise (e.g., are not connected to) a photoswitchable group, and optionally, a sterol.
Statement 7. A nanovesicle according to Statement 6, wherein the photoswitchable glycerophospholipid is present at a concentration of 0.1-80 mol %, including all 0.1 values and ranges therebetween (e.g., 10 mol %).
Statement 8. A nanovesicle according to Statement 6 or Statement 7, wherein the one or more glycerophospholipid is present at a concentration of 5-99.9 mol %, including all 0.1 values and ranges therebetween.
Statement 9. A nanovesicle according to any one of Statements 6-8, wherein the sterol is present a concentration of 1 to 70 mol %, including all 0.1 values and ranges therebetween.
Statement 10. A nanovesicle according to Statement 9, wherein the sterol is chosen from cholesterol, sitosterol, stigmasterol, cholestanol, and the like, and combinations thereof.
Statement 11. A nanovesicle according to any one of Statement 6-10, further comprising one or more cargo molecules.
Statement 12. A nanovesicle according to Statement 11, wherein the one or more cargo molecules are pharmaceutical drugs (e.g., the pharmaceutical drug has a pKₐ of greater than 7 (e.g., is a weak base)).
Statement 13. A nanovesicle according to Statement 11 or Statement 12, wherein the one or more cargo molecules are chosen from doxorubicin, nucleic acids (e.g., siRNA, mRNA, plasmids, and the like, and combinations thereof), peptides, and the like, and combinations thereof.
Statement 14. A nanovesicle according to any one of Statements 6-13, wherein the nanovesicles have a longest linear dimension (e.g., a diameter) of 10-150 nm, including every 0.1 nm value and range therebetween.
Statement 15. A composition comprising one or more nanovesicles according to any one of Statements 6-14 and one or more pharmaceutically acceptable carriers.
Statement 16. A composition according to claim 15, further comprising one or more cargo molecules.
Statement 17. A composition according to Statement 16, wherein the one or more cargo molecules are pharmaceutical drugs (e.g., the pharmaceutical drug has a pKₐ of greater than 7 (e.g., is a weak base)).
Statement 18. A composition according to Statement 16 or Statement 17, wherein the one or more cargo molecules are chosen from doxorubicin, nucleic acids (e.g., siRNA, mRNA, plasmids, and the like, and combinations thereof), peptides, and the like, and combinations thereof.
Statement 19. A method of delivering cargo molecules of one or more nanovesicles according to any one of Statements 11-14 to an individual comprising: administering to the individual a composition comprising the nanovesicles, such that the nanovesicles are delivered to a target site; exposing the target site of the individual to red spectral light, wherein the one or more cargo molecules are released from the one or more nanovesicles and the cargo molecules are delivered to the target site of the individual.
Statement 20. A method according to Statement 19, wherein the one or more cargo molecules are pharmaceutical drugs (e.g., the pharmaceutical drug has a pKₐ of greater than 7 (e.g., is a weak base)).
Statement 21. A method according to Statement 20, wherein the one or more cargo molecules are chosen from doxorubicin, nucleic acids (e.g., siRNA, mRNA, plasmids, and the like, and combinations thereof), peptides, and the like, and combinations thereof.
Statement 22. A method according to any one of Statements 19-21, wherein the exposure (e.g., the red spectral light of the exposure) is produced by a scope (e.g., scope comprising a light source, such as, for example, a laser).

The following examples are presented to illustrate the present disclosure. They are not intended to be limiting in any matter.

### EXAMPLE 1

The following in an example describing the synthesis and use of photoswitchable glycerophospholipids and nanovesicles formed therefrom.

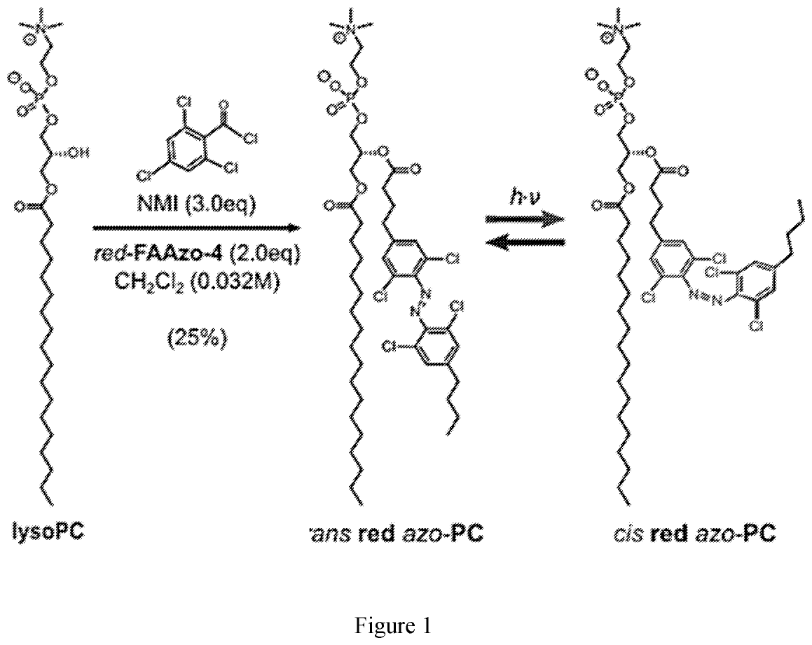

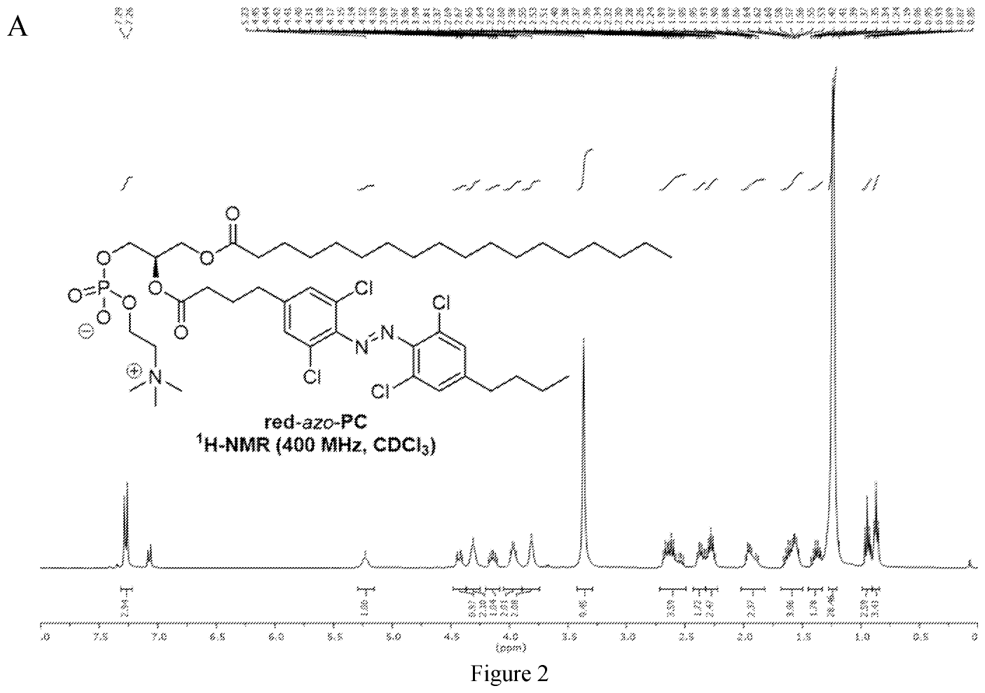

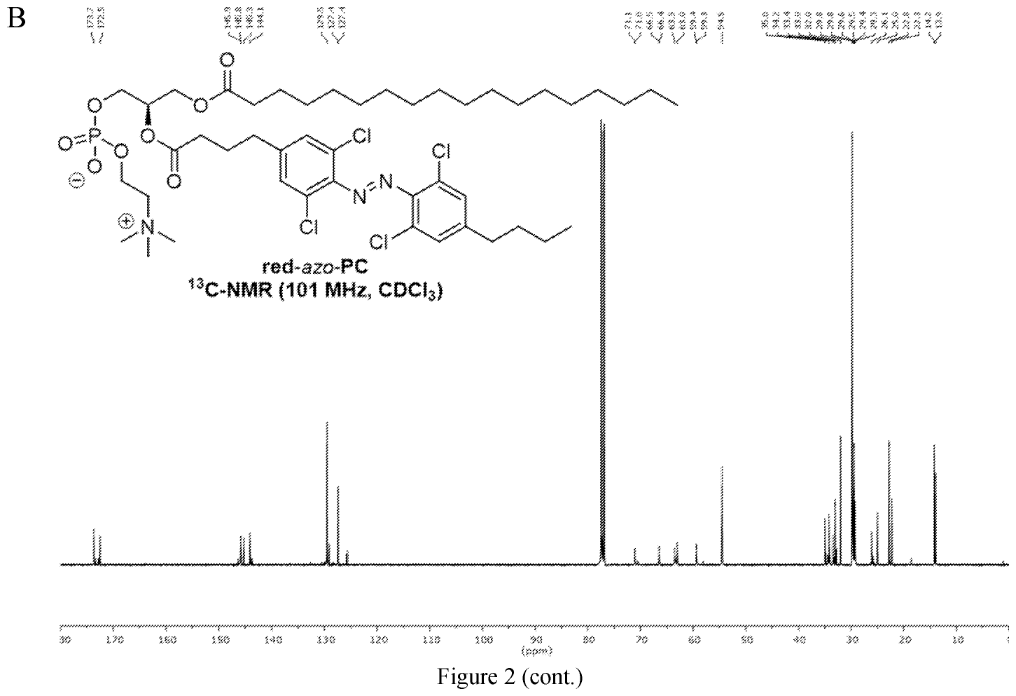

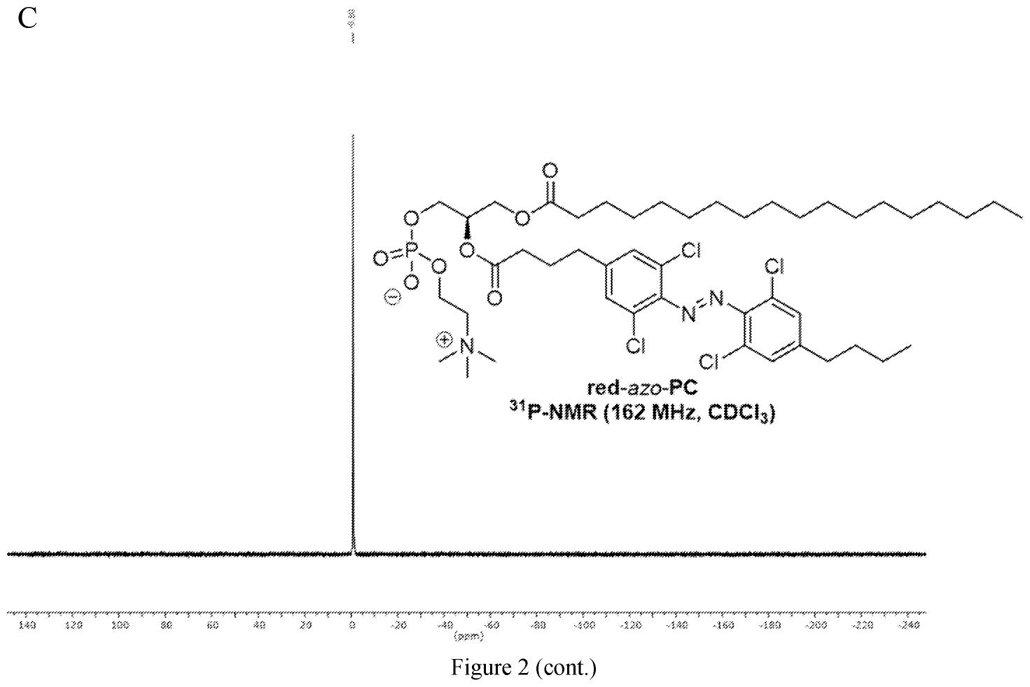

Synthesis of red azo-PC and vesicle preparation. The photolipids were synthesized as follows (FIG. 1): First 1-steaoryl-sn-glycero-3-phosphocholine (lysoPC) was prepared by a selective sn1-monoacylation of L-α-glycerylphosphorylcholine (α-GPC) in 70% yield. A Yamaguchi esterification protocol was used to install the red-shifted photoswitchable fatty acid red-FAAzo-4 at the sn2 position. The red-FAAzo-4, which contains a tetra-ortho-chlorinated azobenzene group, was synthesized according to a previous protocol. By this approach, red azo-PC was obtained in 25% yield.

After purifying the lipids, a protocol to prepare small (pSUVs) and giant (pGUVs) unilamellar vesicles from red azo-PC was devised to investigate the photoisomerization process in a membrane setting. The small vesicles with a diameter of ˜100 nm were prepared according to a previously established ultra-sonication protocol. The pGUVs (diameter of ˜5 μm to ˜100 μm) were prepared by mixing 99 mol % red azo-PC with 1 mol % of a red fluorescent dye (either TexasRed-DHPE or Atto633-DPPE) using electroformation.

### Chemical Synthesis and Characterization of red-azo-PC

Nuclear magnetic resonance (NMR) spectroscopy: NMR spectra were acquired with the following spectrometers: Varian INOVA 400 (400 MHz for ¹H and 101 MHz for ¹³C spectroscopy) and Bruker Avance III HD 400 with Cryo-head (400 MHz for ¹H and 101 MHz for ¹³C spectroscopy). Chemical shifts (δ) are reported in parts per million (ppm) relative to tetramethylsilane (TMS). The deuterated solvent CDCl₃ was used as internal references. Spin multiplicities are described as follows: s (singlet), d (doublet), t (triplet), q (quartet), quint (quintet), m (multiplet), br (broad) or a combination thereof. Structural analysis was conducted with ¹H- and ¹³C-NMR spectra with the aid of additional 2D spectra (COSY, HMBC, HSQC, NOESY). Spectra analysis was conducted with the software MestReNova v. 10.0.1-14719. The ³¹P-NMR spectra were referenced using the ¹H-NMR spectra of the same compounds as an absolute reference.

Mass spectrometry (MS): The high resolution MS spectra were recorded on a Thermo Finnigan LTQ FT (ESI: electrospray ionization).

Infrared spectroscopy (IR): IR spectra were recorded on a PerkinElmer Spectrum BX II FT-IR device equipped with an attenuated total reflection (ATR) measuring unit. For measurements, the neat substances were directly applied as a thin film on the ATR unit. The measured wavenumbers are reported with their relative intensities which were classified as: vs (very strong), s (strong), m (medium), w (weak), vw (very weak), br (broad) or combinations thereof.

Chemical Methods: Unless otherwise noted, all reactions were magnetically stirred under inert gas (N₂) atmosphere using standard Schlenk techniques. Glassware was evacuated and dried by heating with a heat-gun (set to 550° C.). Cannulas and syringes were used for the transfer of reagents or solvents which were flooded with inert gas (3×) before use. Purification by column chromatography was performed under elevated pressure (flash column chromatography) on Geduran® Si60 silica gel (40-63 μm) from Merck KGaA. After flash column chromatography, the concentrated fractions were filtered once through a glass frit. Silica gel F₂₅₄ TLC plates from Merck KGaA were used for monitoring reactions, analyzing fractions of column chromatography and measuring Rr values. To visualize the analytes, TLC plates were irradiated with UV light and/or appropriate staining solutions and subsequent heating. Freeze-drying refers to freezing of the respective sample in liquid nitrogen followed by evacuating the containing flask with high vacuum (<1 mbar) and slow thawing to rt.

Chemicals: All chemicals were purchased from Sigma Aldrich, Fisher Scientific, TCI Europe, Chempur, Alfa Aesar or Acros Organics. Solvents purchased in technical grade quality and were distilled under reduced pressure and used for purification procedures. Purchased solvents in HPLC and analytical grade quality were used without further purification. Unless otherwise noted, reactions were performed using dry solvents. Dichloromethane (CH₂Cl₂) was dried by distillation from CaH₂. All other reagents with a purity of >95% were purchased from commercial sources and used without further purification. For running extra dry reactions with synthetic compounds, stock solutions were prepared in PhMe, the respective amounts transferred into dried glassware and the solvent was removed by stirring under high vacuum (<1 mbar). This procedure was followed by freeze-drying the compound to ensure that H₂O was fully removed. The CAM staining solution was prepared by dissolving (Ce(NH₄)₂(NO₃)₆ (0.5 g) and (NH₄)₆Mo₇O₂₄ 4H₂O (48 g) in H₂O (940 mL) and adding conc. H₂SO₄ (60 mL).

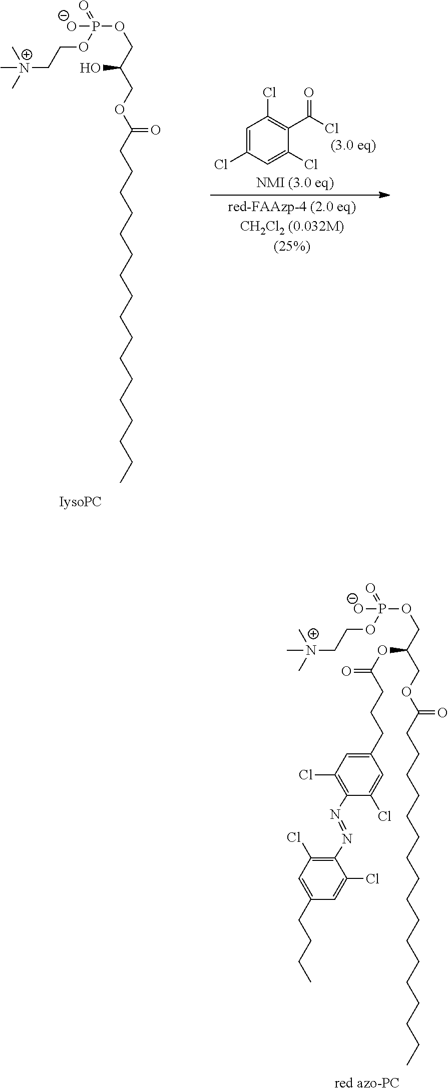 [SMILES: CCCCCCCCCCCCCCCCCC(=O)OC[C@@H](O)COP(=O)([O-])OCC[N+](C)(C)C ; O=C(Cl)c1c(Cl)cc(Cl)cc1Cl ; CCCCCCCCCCCCCCCCCC(=O)OC[C@H](COP(=O)([O-])OCC[N+](C)(C)C)OC(=O)CCCc1cc(Cl)c(/N=N/c2c(Cl)cc(CCCC)cc2Cl)c(Cl)c1]

Red-FAAzo-4 (468.7 mg, 0.991 mmol, 2.0 eq) was dissolved in CH₂Cl₂ (5.5 mL) and NMI (118 μL, 1.49 mmol, 3.0 eq) as well as 2,4,6-trichlorobenzoyl chloride (213 μL, 1.49 mmol, 3.0 eq) was added dropwise. The mixture was transferred by dropwise addition to lysoPC (259.4 mg, 0.495 mmol, 1.0 eq) in CH₂Cl₂ (10.0 mL). After stirring for 20 hours at room temperature, the solution was directly subjected to purification by three-fold flash column chromatography (CH₂Cl₂:MeOH:H₂O=7.5:2.5:0.2→7:3:0.2) to give (R)-2-((4-(4-((4-butyl-2,6-dichlorophenyl)diazenyl)-3,5-dichlorophenyl)butanoyl)oxy)-3-(stearoyloxy)propyl (2-(trimethylammonio)ethyl) phosphate (red-azo-PC) (117.6 mg, 0.122 mmol, 25%) as a red gum.

R_{f}(CH₂Cl₂:MeOH:H₂O=10:4:0.5)=0.46. (visible,CAM)

¹H NMR (400 MHz, CDCl₃) δ (ppm)=7.27 (d, J=10.4 Hz, 4H), 5.23 (br s, 1H), 4.48-4.37 (m, 1H), 4.31 (s, 2H), 4.13 (dt, J=12.2, 6.1 Hz, 1H), 3.97 (q, J=6.6 Hz, 2H), 3.81 (s, 2H), 3.37 (s, 9H), 2.72-2.49 (m, 4H), 2.43-2.32 (m, 2H), 2.28 (p, J=7.4 Hz, 2H), 2.01-1.84 (m, 2H), 1.68-1.49 (m, 4H), 1.39 (p, J=7.4 Hz, 2H), 1.24 (br s, 28H), 0.95 (t, J=7.3 Hz, 3H), 0.87 (t, J=6.7 Hz, 3H).

¹³C NMR (101 MHZ, CDCl₃) δ (ppm)=173.7, 172.5, 145.9, 145.8, 145.3, 144.1, 129.5, 127.4, 127.4, 71.1 (d, J=7.0 Hz), 66.4 (d, J=5.9 Hz), 63.5, 63.0, 59.4 (d, J=4.0 Hz), 54.5, 35.0, 34.2, 33.4, 33.0, 32.0, 29.8, 29.8, 29.6, 29.5, 29.4, 29.3, 26.1, 25.0, 22.8, 22.3, 14.2, 13.9.

³¹P NMR (162 MHz, CDCl₃) δ (ppm)=−0.80.

HRMS (ESI): calc. for C₄₆H₇₃Cl₄N₃O₈P⁺ [M+H⁺]⁺: 968.3854, found: 968.3854.

IR (Diamond-ATR, neat) υₘₐₓ (cm⁻¹)=3366 (br, w), 2956 (w), 2923 (s), 2852 (m), 1732 (s), 1590 (w), 1550 (w), 1466 (m), 1400 (m), 1378 (w), 1339 (w), 1235, (s), 1177 (m), 1145 (m), 1088 (vs), 1063 (vs), 969 (s), 925 (m), 809 (vs), 721 (s).

### EXAMPLE 2

The following in an example describing the synthesis and use of photoswitchable glycerophospholipids and nanovesicles formed therefrom.

Described herein is the incorporation of UV-A or red-light photoswitchable phosphatidylcholine analogs (AzoPC and redAzoPC) in conventional LNPs to generate photoactivatable LNPs (paLNPs) having comparable structural integrity, drug loading capacity, and size distribution to the parent DSPC-cholesterol liposomes. The nanoparticles exhibited 65-70% drug release (doxorubicin), which could be induced from these systems by irradiation with pulsed light based on trans to cis azobenzene isomerization. It was confirmed in vitro that paLNPs are non-toxic in the dark but convey cytotoxicity upon irradiation in a human cancer cell line. In vivo studies in zebrafish embryos demonstrated prolonged blood circulation and extravasation of paLNPs comparable to clinically approved formulations, with enhanced drug release following irradiation with pulsed light. Conclusively, paLNPs closely mimic the properties of clinically approved LNPs with the added benefit of light-induced drug release making them promising candidates for clinical development.

Described herein is a liposomal light-triggered release system containing photoswitchable phosphatidylcholine analogs with azobenzenes incorporated into the lipid tail, compounds termed AzoPC and red-AzoPC. This strategy enabled the design of an exterior lipid composition that allows long circulation lifetimes, incorporation of an agent responsive to light, and an aqueous interior offering small molecule drugs to be encapsulated. It was shown that DSPC-cholesterol systems incorporating AzoPC (in the trans-form) at low (10 mol %) levels result in liposomes that have similar size and drug (doxorubicin) loading properties as parent LNPs. When stimulated to adopt the cis-form, the AzoPC containing liposomes exhibited triggered release properties resulting in enhanced cytotoxic effects in vitro. The responsiveness of photoactivatable LNPs (paLNPs) to a different wavelength is tuned by substituting AzoPC with a red-shifted variant red-AzoPC. In vivo studies confirmed long blood circulation half-lives and triggered release properties of paLNP system. These data demonstrated the therapeutic utility of liposomal systems containing AzoPC.

### Results & Discussion

Design of Doxorubicin-Loaded paLNP Liposomes.

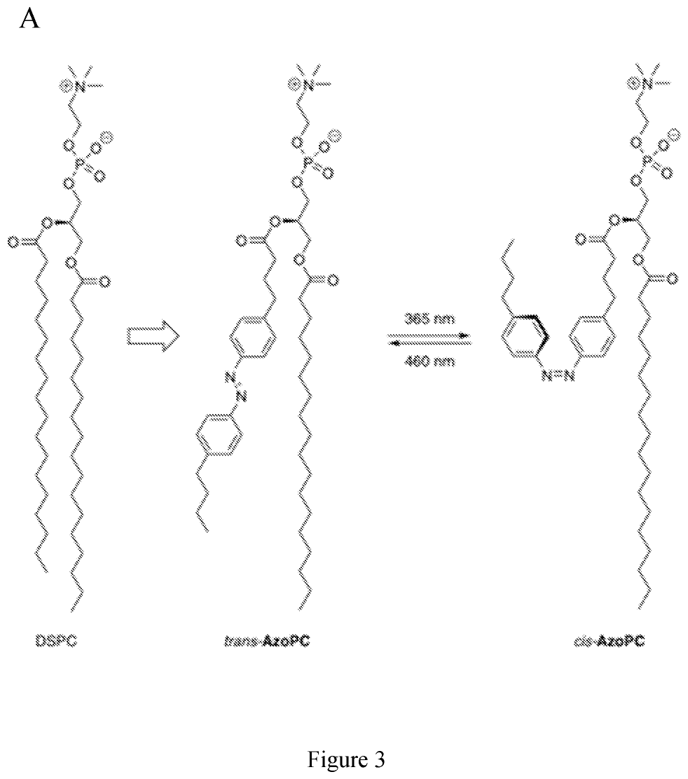

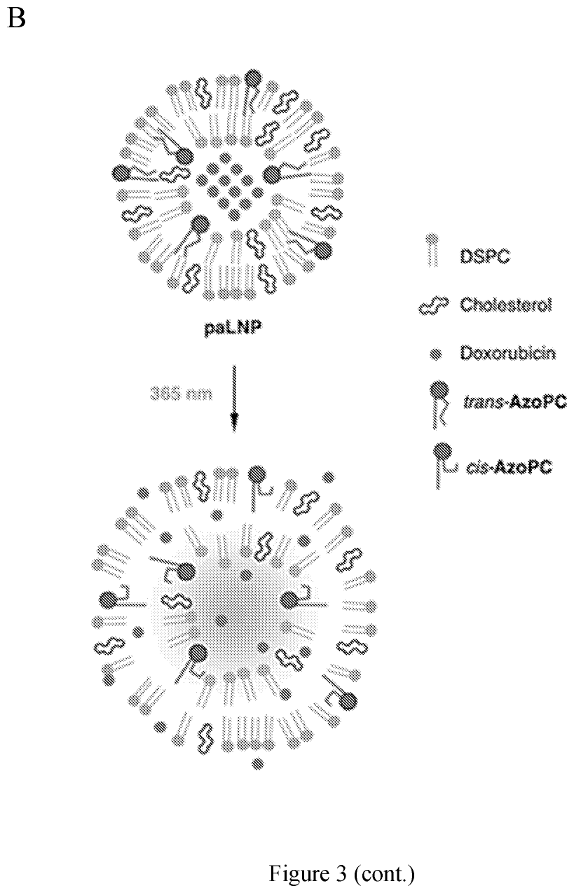

In order to design a light-triggered release system enabling efficient drug loading and long-circulation properties, conventional DSPC-cholesterol liposomes (55 mol % DSPC, 45 mol % Chol) were modified by incorporating a UV-A photoswitchable AzoPC (FIG. 3A). Stimulation of paLNPs at 365 nm should trigger trans to cis isomerization resulting in doxorubicin release (FIG. 3B).

Synthesis and Characterization of Doxorubicin-Loaded paLNP Liposomes.

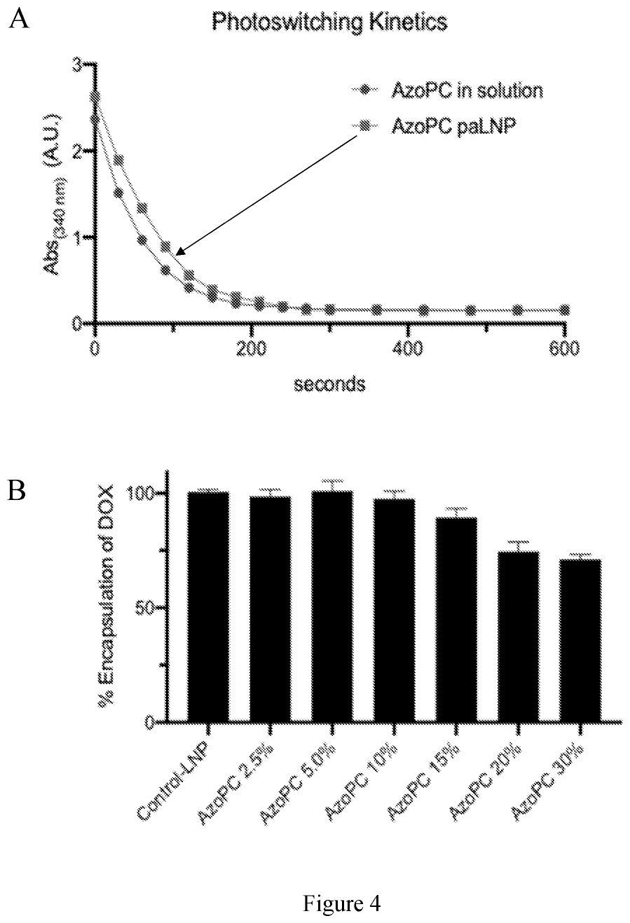

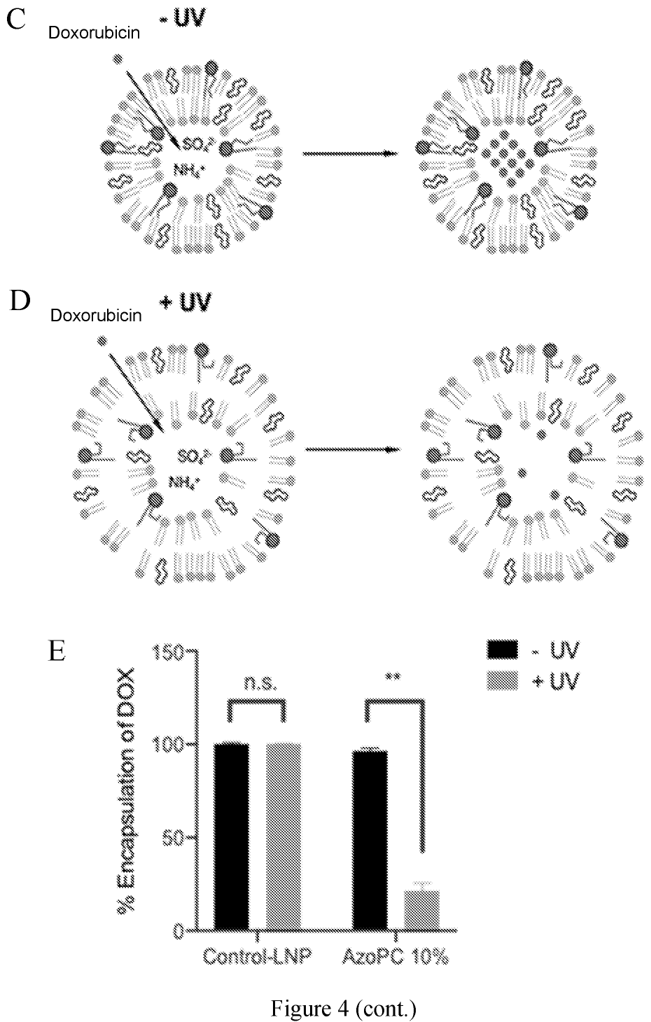

DSPC-cholesterol liposomes were prepared employing ethanol dilution-rapid mixing techniques and subsequent dialysis steps to contain 300 mM ammonium sulfate in their aqueous core and phosphate buffered saline (PBS) as an exterior buffer and were used as control formulations (Control-LNP). A range of paLNP liposomes were synthesized by titrating varying amounts of AzoPC (2.5, 5, 10, 15, 20, 30 mol %) into the control DSPC-cholesterol liposomes where the added AzoPC substituted for DSPC. Dynamic light scattering (DLS) analysis of the particles showed a monodisperse population (PDI<0.1) of uniformly sized ˜55-60 nm particles (Table 1). The trans to cis photoswitching kinetics of AzoPC incorporated into paLNPs was comparable to that observed in the stock ethanol solution as observed by measuring absorbance at 340 nm at 30 s intervals (λ=365 nm) (FIG. 4A).

TABLE 1
Physicochemical characterization of LNPs. Hydrodynamic
diameter and size distribution (polydispersity
index, PDI) of Control-LNP (DSPC-Chol system) and
paLNPs containing various amounts of AzoPC.

| Lipid composition | Mean diameter ± SD (nm) | PDI |
|---|---|---|
| Control-LNP | 52.83 ± 2.588 | 0.038 |
| AzoPC 2.5% | 55.49 ± 0.732 | 0.064 |
| AzoPC 5% | 56.33 ± 1.811 | 0.083 |
| AzoPC 10% | 57.89 ± 1.150 | 0.072 |
| AzoPC 15% | 59.20 ± 1.689 | 0.097 |
| AzoPC 20% | 59.86 ± 4.118 | 0.105 |
| AzoPC 30% | 63.44 ± 2.988 | 0.101 |

The LNP formulations were loaded with doxorubicin using the pH gradient (interior acidic) generated by encapsulated ammonium sulphate. The LNP were incubated at 65° C. for 30 min with free drug to achieve a maximum encapsulated drug:lipid ratio (wt/wt) of 0.1 after which unentrapped doxorubicin was removed via dialysis. Drug:lipid ratios were assayed for samples taken before and after drug loading and used to calculate percent entrapment. Efficient loading was achieved for liposomes containing up to 10% Azo-PC. Formulations containing higher mol % of AzoPC (15-30 mol %) were unable to maintain the ammonium sulphate gradient effectively, which resulted in lower drug loading (FIG. 4B). The dependence of drug loading efficiency on the ammonium sulphate gradient in the paLNP system was confirmed by switching the AzoPC conformation to the cis form prior to drug loading which resulted in a significant reduction in entrapment efficiency in cis-paLNPs (˜20% entrapment) as compared to trans-paLNPs (˜100% entrapment) (FIG. 4C/D). The control LNP did not show any change in encapsulation efficiency upon irradiation (FIG. 4E).

Liposomes containing 10 mol % AzoPC exhibit up to 80% light-triggered release of doxorubicin when AzoPC is switched to the cis form.

Drug release from paLNPs following irradiation was measured in PBS at room temperature. paLNPs loaded with doxorubicin at a drug:lipid ratio of 0.1 (wt/wt) and a concentration of 3 mg/mL total lipid were irradiated with the UV-A light source (365 nm) for 5 min followed by storage in the dark for 1 h. Samples were assayed for drug release by measuring absorbance at 492 nm. Limited release was observed in paLNPs containing 2.5-5 mol % AzoPC while paLNPs containing 10-30 mol % AzoPC showed up to 20% drug release at the 1 h timepoint (FIG. 3A). paLNP containing 10% AzoPC in the trans form showed drug loading and drug release properties that were similar to the control LNP system (DSPC-cholesterol), whereas paLNP systems with higher trans AzoPC contents exhibited decreased drug loading capabilities that may be attributed to increased permeability of the liposome bilayer. paLNP systems containing 10 mol % AzoPC were used for subsequent experiments.

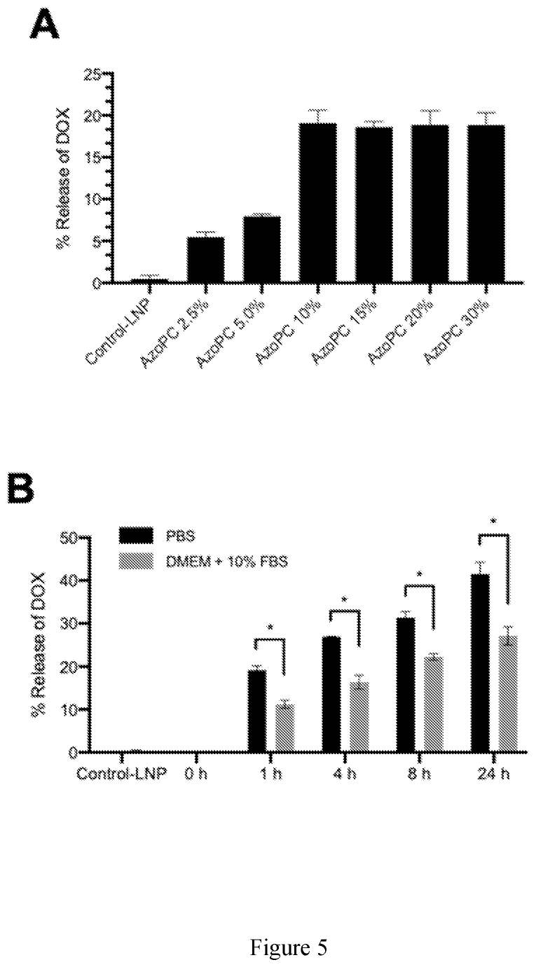

Initial work was performed to determine whether triggered release could be achieved in response to irradiation to switch the azo-PC from the trans to the cis form. It was found that significant triggered release of up to 40% could be achieved for paLNP containing 10 mol % Azo-PC following 5 min irradiation at 365 nm at room temperature when the paLNP were suspended in PBS (FIG. 5B). Next, it was tested whether light triggered release was influenced by the presence of serum proteins. It is well known that serum proteins adsorb to liposomal surfaces forming a protein corona that can influence membrane permeability and other properties such as accessibility of light to membrane surface. Light-triggered release from paLNPs containing 10 mol % AzoPC following dilution into cell culture medium (DMEM) containing serum (10% FBS) was evaluated. It was found that the presence of serum proteins inhibited light-triggered drug release significantly compared to paLNP in PBS. After an initial irradiation time of 5 min at 365 nm followed by storage in the dark, approximately 25% of doxorubicin was released after a 24 h time period as compared to 40% of doxorubicin released in PBS (FIG. 5B).

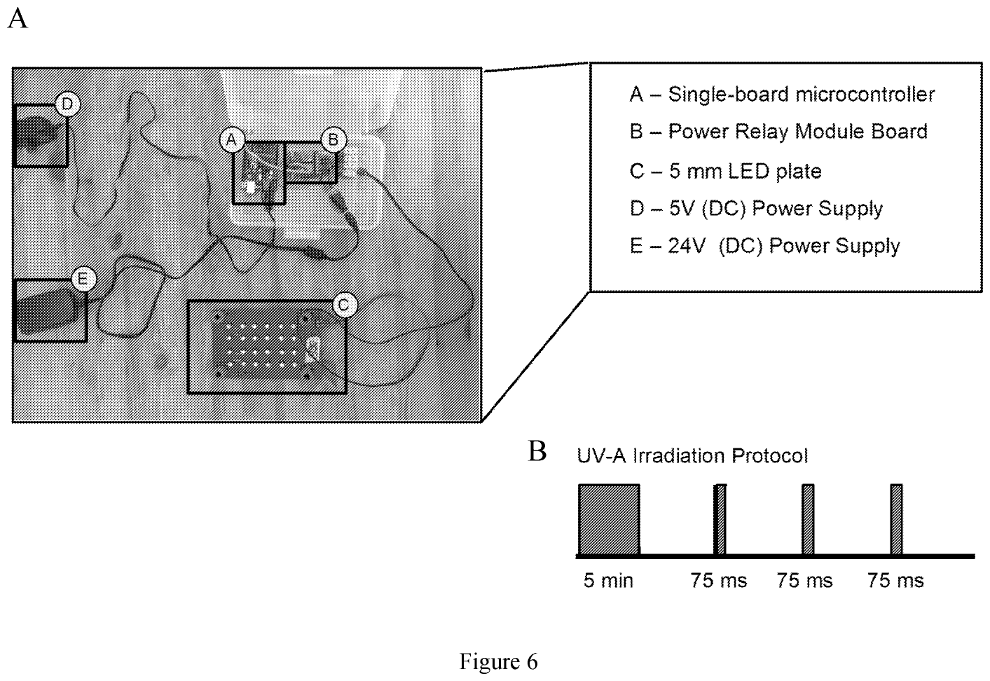

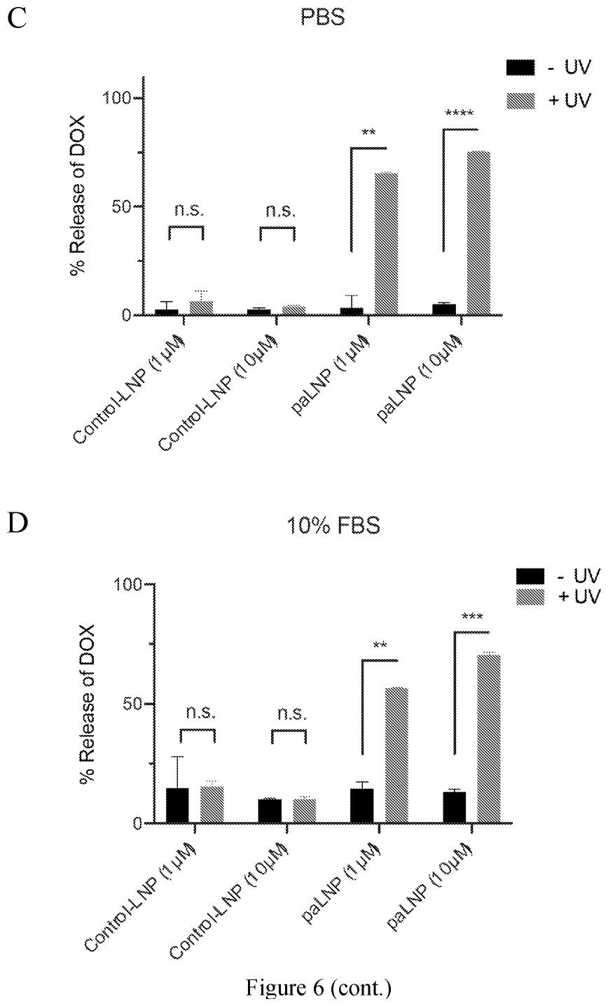

Of note, AzoPC in the cis form will spontaneously revert to its trans form over time, potentially reducing leakage from the irradiated paLNP. It was investigated whether the amount of drug release could be increased through pulsed irradiation following the 5 min initial irradiation to prevent re-isomerization keeping the AzoPC in its cis form for a longer period of time. The paLNP were subjected to pulses of LED light (wavelength 365 nm) of 75 ms duration every 15 s over a 24 h period (FIG. 6A-B). It was found that this pulsed irradiation protocol led to increased drug release within 24 h compared with initial irradiation. This result could be explained by thermal relaxation and repeated switching over time. While constant irradiation over the same time frame could potentially produce a similar effect in terms of release, the pulsed method allows for successful switching and maintenance of the AzoPC in cis conformation at >100 times less irradiation time, preventing any side effects associated with prolonged exposure to UV-A light such as increase in temperature of irradiated area or cytotoxicity.

Correspondingly, the in vitro drug release experiments were repeated on paLNP in PBS and DMEM, where the initial irradiation time of 5 min was followed by pulsed irradiation. This pulsed irradiation enabled improved drug release of 75-80% from paLNPs in PBS (FIG. 6C) and 65-70% from paLNPs in serum containing medium (DMEM with 10% FBS) (FIG. 6D). The control LNP demonstrated a relatively low doxorubicin release and did not show any change in drug release on irradiation (FIG. 6C/D).

The morphology of doxorubicin-loaded liposomes containing AzoPC following pulsed irradiation is consistent with drug release.

Liposomes loaded with doxorubicin employing pH loading techniques exhibit characteristic “coffee bean” morphology as detected by cryo-TEM due to the formation of nano-sized crystals of precipitated doxorubicin in the centre of the liposomes. It is of interest to determine whether similar morphology is exhibited by the loaded paLNP and whether this morphology is affected by the light-triggered release of doxorubicin. We performed structural evaluation of the paLNP formulation using cryo-TEM imaging.

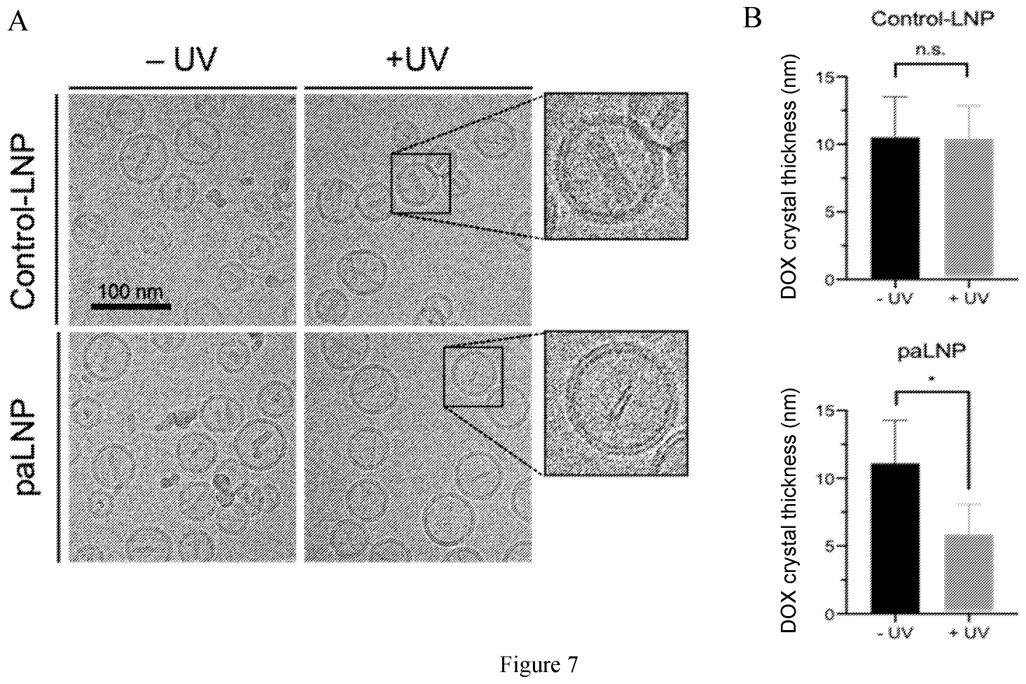

As shown in FIG. 7A, paLNPs showed a typical bilayer structure, indistinguishable from the control DSPC-cholesterol liposomes, with sizes in agreement with those obtained via DLS (˜55-60 nm). Control-LNPs and paLNPs loaded with doxorubicin showed doxorubicin crystalized within the liposome interior. After light-triggered release using pulsed irradiation at 365 nm over 24 h, there is a clear decrease in the number of entrapped drug crystals within the paLNPs, while the control DSPC-cholesterol liposomes show no visible changes. This was also confirmed through quantification of crystal thickness within the various samples (FIG. 7B).

Doxorubicin released from loaded paLNP following irradiation is biologically active.

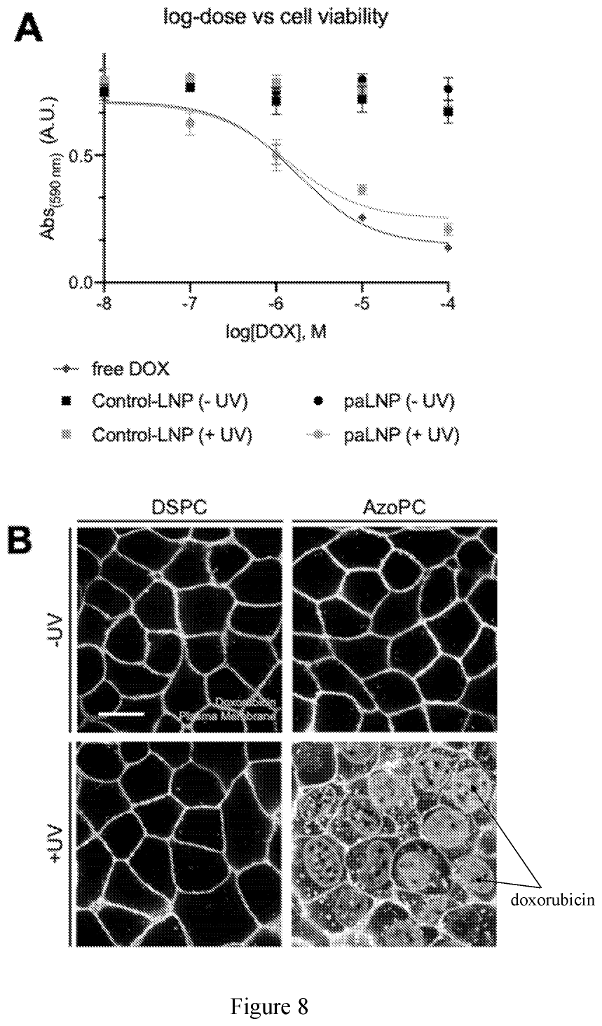

Doxorubicin is a cytotoxic agent and its release from paLNP following irradiation would be expected to result in cytotoxic effects on nearby tissues. In order to demonstrate this, the effects of light-released doxorubicin on the viability of a human derived liver cancer cell line (i.e., HuH7 cells) was investigated in vitro. The cell viability effects of doxorubicin were compared in its free vs liposome encapsulated forms with and without light-triggered drug release. HuH7 cells were treated with either free doxorubicin (dissolved in PBS), doxorubicin-loaded Control-LNP or paLNP at drug concentrations up to 100 μM. Cells were subjected to irradiation at 365 nm for 5 min at 6 h post exposure to trigger drug release, followed by pulsed irradiation at 365 nm for 24 h to keep the AzoPC in its cis form. As expected, Control-LNP (with or without UV irradiation) did not result in a decrease in viable cells due to their limited drug release properties. In contrast, treatments using paLNPs were highly dependent on the light-trigger. Whereas paLNP without UV irradiation did not affect cell viability, photoswitched paLNP resulted in a dose-dependent decrease in the number of viable cells 24 h after treatment similar to that of free doxorubicin (FIG. 8A). To confirm the significant light-triggered release of doxorubicin from paLNP, HuH7 cells were treated with Control-LNP or paLNP at a doxorubicin concentration of 10 μM as stated above and analyzed through confocal microscopy with and without UV treatment. The confocal images demonstrate that only paLNPs result in effective release of doxorubicin after irradiation. Once released, doxorubicin accumulates in the nucleus and is fluorescent. (FIG. 8B).

Formulation and Characterization of Red-Shifted paLNP Incorporating Tetra-Ortho-Chloro-Azobenzene Modified AzoPC.

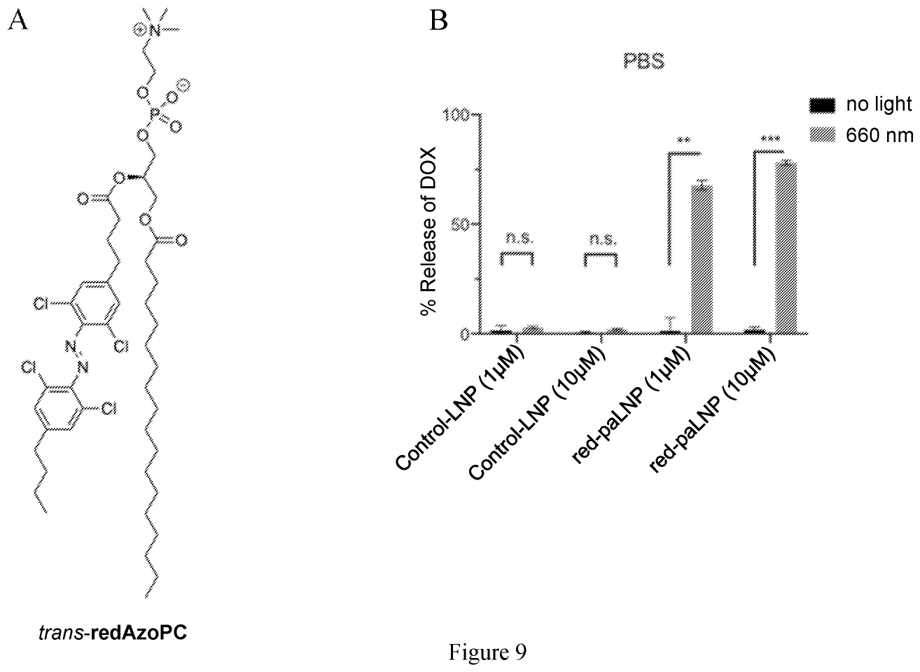

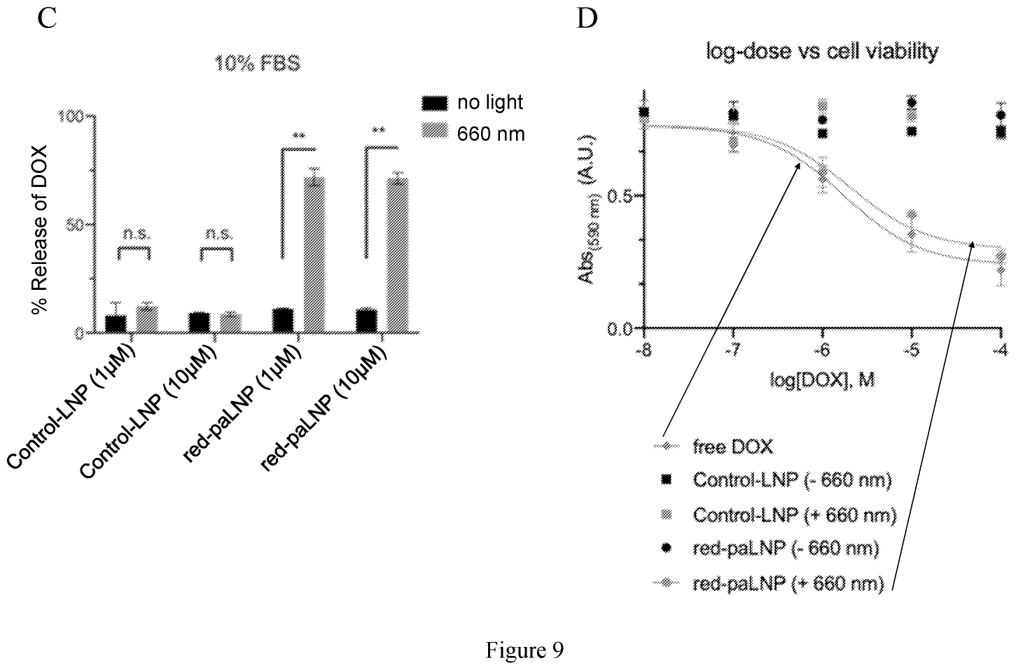

Incorporation of AzoPC sensitive to 365 nm could be perceived as a challenge for in vivo translation due to the limitation in tissue penetration depth and low tolerance to UV-A light. To extend the applicability of our light-triggered release system, we developed a red-shifted version of AzoPC, termed redAzoPC, that undergoes a switch from the cis to the trans conformation at 660 nm (FIG. 9A). Red-paLNPs were prepared using the previously established procedures by adding 10 mol % redAzoPC into the control DSPC-cholesterol liposomes (substituting DSPC for redAzoPC). Characterization of the red-paLNP particles showed a monodisperse population (PDI <0.1) of uniformly sized ˜56-58 nm nanoparticles with >98% entrapment efficiency of doxorubicin, matching the physicochemical characteristics of control and paLNPs.

Drug release from red-paLNPs following irradiation was measured in PBS and cell culture medium (DMEM) containing serum (10% FBS) at room temperature. The red-paLNP were subjected to pulses of LED light (wavelength 660 nm) of 75 ms duration every 15 s over a 24 h period. This pulsed irradiation enabled a 75-80% doxorubicin release from red-paLNPs in PBS (FIG. 9B) and 70-75% release from red-paLNPs in serum containing medium (FIG. 9C). In contrast, control-LNPs did not show any change in drug release following deep-red light irradiation (FIG. 9B/C).

To assess the effects of doxorubicin in its free vs. red-paLNP encapsulated forms (with and without light-trigger) on the viability of a human derived liver cancer cell line (i.e. HuH7 cells), an in vitro study was performed. HuH7 cells were treated with either free doxorubicin (dissolved in PBS), doxorubicin-loaded Control-LNP or red-paLNP at drug concentrations up to 100 μM. Cells were subjected to irradiation at 660 nm for 5 min at 6 h post exposure to trigger drug release, followed by pulsed irradiation at 660 nm for 24 h to keep the redAzoPC in its cis isoform. As seen previously, Control-LNP (with or without irradiation) did not result in a decrease in viable cells due to their limited drug release properties. In contrast, cytotoxic effects of red-paLNPs were highly dependent on the light-trigger. Similar to the results seen with the paLNP (UV-A light), the red-paLNP did not affect cell viability without irradiation whereas the photoswitched red-paLNP resulted in a dose-dependent decrease in the number of viable cells 24 h after treatment similar to that of free doxorubicin (FIG. 9D).

paLNP and red-paLNP Display Long Circulation Lifetimes In Vivo Following i.v. Administration.

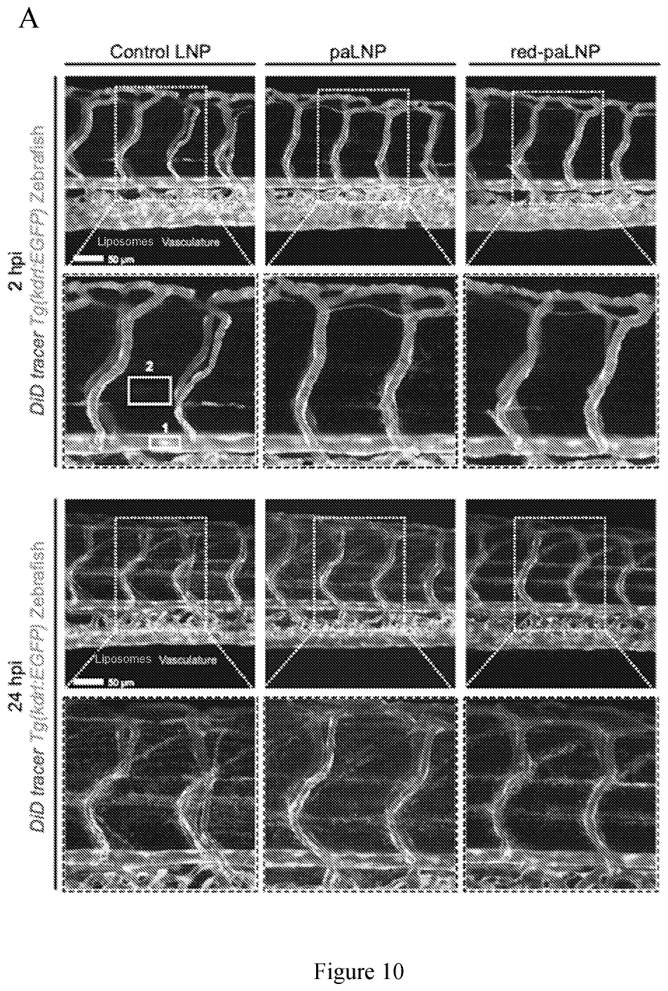

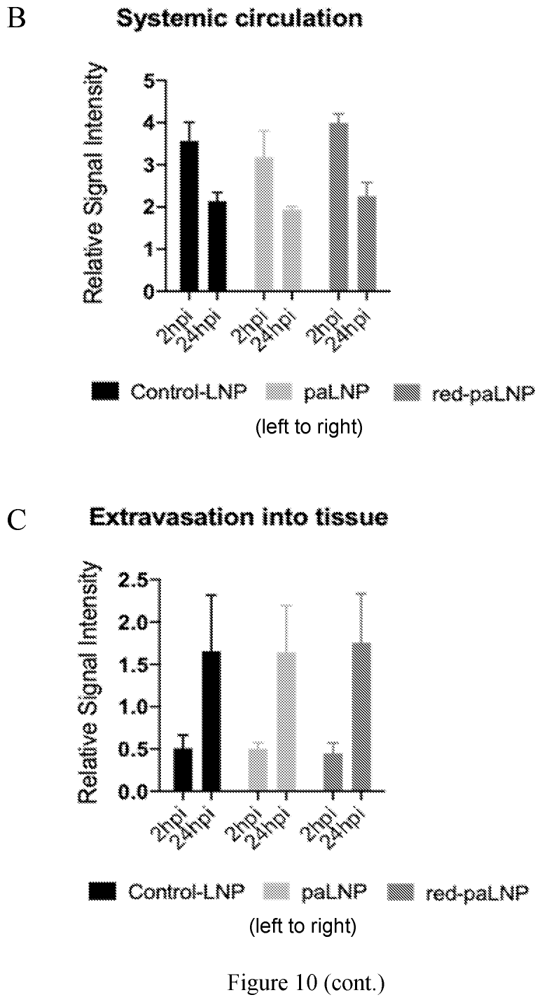

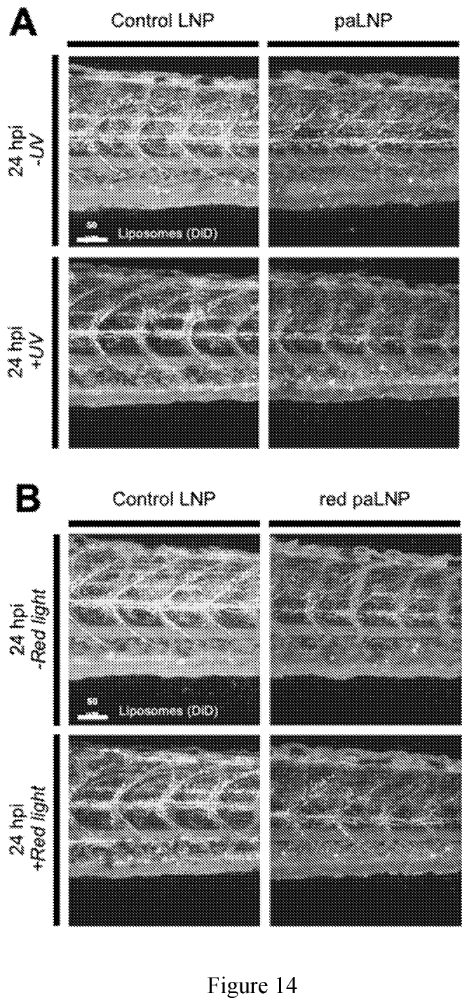

To assess the pharmacokinetic properties of developed paLNP systems (i.e. influence of incorporating AzoPC analogs into conventional liposomes), the zebrafish embryo model was used. Zebrafish embryos are a reliable and predictive in vivo tool to investigate liposomal circulation behavior and clearance mechanisms. First, fluorescently labeled Control-LNP, paLNP, and red-paLNP (1 nL) were intravenously injected into transgenic zebrafish expressing green fluorescent protein in their vascular endothelial cells (Tgkdrl:EGFP) at total lipid concentrations of 10 mg/mL. Next, we performed confocal microscopy imaging of the tail region was performed 2 h and 24 h post-injection (hpi). All liposomal systems (with and without irradiation) demonstrated excellent circulation properties within blood vessels 2 hpi (FIG. 10A/B and FIG. 14) without any signs of agglomeration within the intersegmental vessels (ISV) and dorsal longitudinal anastomotic vessels (DLAV). At 24 hpi, significant extravasation of LNPs into surrounding tissue and accumulations in the posterior caudal vein (PCV) region (indicating macrophage clearance) was observed (FIG. 8A/C). These pharmacokinetic characteristics, i.e. desirable systemic circulation resulting in pronounced tissue extravasation, are typical for long-circulating liposomes. Importantly, incorporation of photoswitchable AzoPC analogs into LNP did not affect the pharmacokinetic properties thereby confirming the ideal characteristics for a light-triggered release system.

Light-Triggered Release of Doxorubicin from paLNP Systems In Vivo.

In assessing the light-triggered release of doxorubicin from paLNPs, various factors must be considered to enable its detection. Upon i.v. injection, doxorubicin is entrapped in circulating LNPs (fluorescence is quenched). Release of doxorubicin into circulation does not result in increased fluorescence due to low quantum efficiency and rapid clearance. Release into tissue however, is detectable due to fluorescence de-quenching and enhanced penetration (fluorescent area). Based on these considerations, we injected Control-LNP, paLNP, and red-paLNP (2 nL) at a doxorubicin concentration of 3 mg/mL into wildtype Tg(abc/tübingen) zebrafish embryos. Following tissue extravasation and accumulation, one set of zebrafish was exposed to pulsed UV-A (365 nm) or deep-red light (660 nm) irradiation for 24 h. Next, confocal microscopy imaging of the tail region was performed and analyzed the penetration area of free doxorubicin.

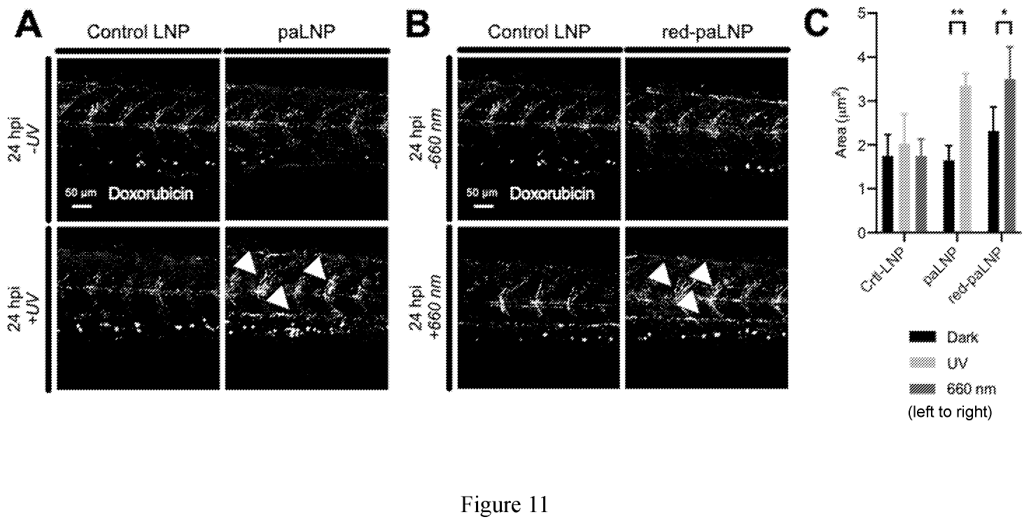

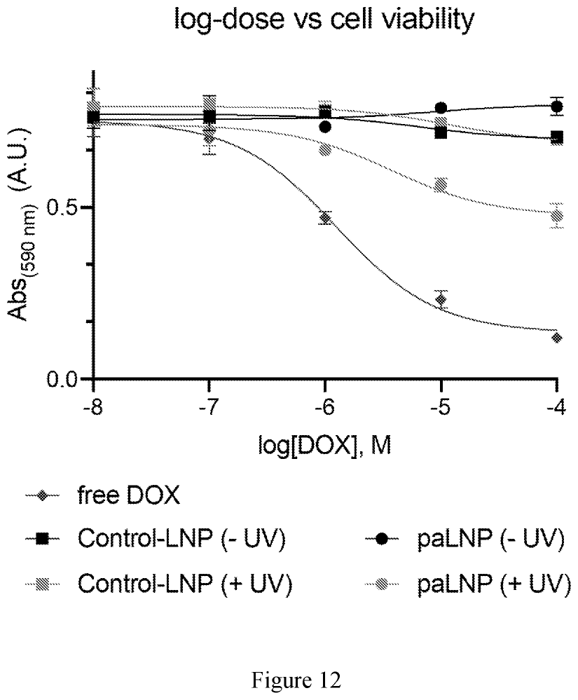

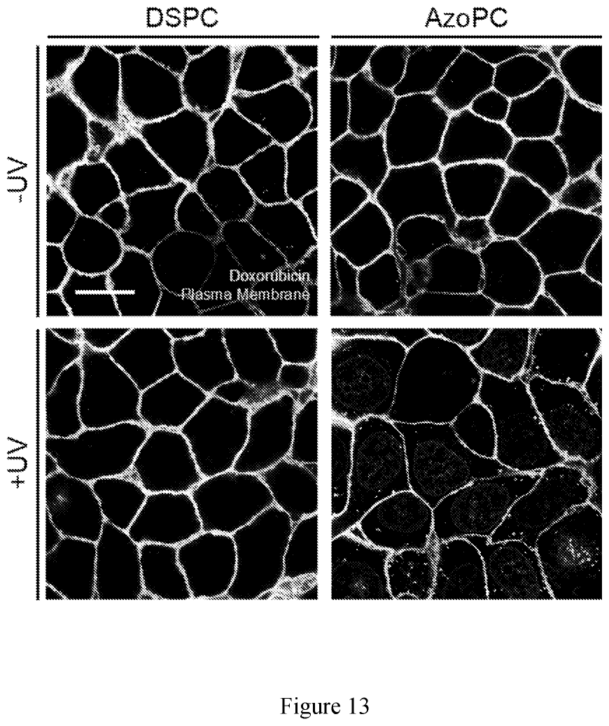

All tested LNPs released doxorubicin 48 hpi (FIG. 11A, B). Although similar doxorubicin signals in the absence of light-trigger were detected for all LNPs, paLNP and red-paLNP triggered with UV-A or deep red pulsed light, respectively, demonstrated significantly enhanced doxorubicin release in zebrafish embryos (FIG. 11C). This result highlights the potential of paLNPs for triggered drug release in vivo.

### Conclusion

These data demonstrate that DSPC-cholesterol liposomes containing 10 mol % photoswitchable phosphatidylcholines (substituting for DSPC) enable light-triggered doxorubicin release in a physiological context. These paLNPs exhibit similar size-distribution, stability, and loading efficiencies as the parent DSPC-cholesterol systems which are clinically approved and widely used in human cancer therapy. They have the added benefit of being able to release contents upon UV-A or deep-red light irradiation inducing a trans to cis isomerization in photoswitchable phosphatidylcholine analogs. This results in up to 80% release of encapsulated doxorubicin over 24 h. The triggered release could potentially be made more rapid in response to a higher intensity light source. The described characteristics in combination with their long blood circulation half-lives make the paLNPs interesting and promising candidates for clinical development.

### Materials and Methods

Materials: Phospholipids used for liposome preparation 1,2-distearolyl-sn-glycero-3-phosphocholine (DSPC) was purchased from Avanti Polar Lipids (Alabaster, AL). Cholesterol and MS-222 (Tricaine) and agarose was purchased from Sigma-Aldrich (Saint Louis, MO). AzoPC and redAzoPC was provided by the lab of Dr. Dirk Trauner (New York University). Ammonium sulphate, Dulbecco's phosphate buffered saline (PBS), fetal bovine serum (FBS) and Triton X-100 were purchased from Sigma-Aldrich (Saint Louis, MO). DOX,

The Cell-DISCO was engineered in the Trauner lab and used as described. A 365 nm/660 nm LED (Roithner Lasertechnik) plate was used at one 75 ms flash per 15 s.

Liposome preparation: Lipid stocks of cholesterol and DSPC were co-dissolved in ethanol at appropriate molar ratios. In some cases, AzoPC or redAzoPC was incorporated into the lipid mix at varying molar ratios, keeping the DSPC to cholesterol ratio constant. All the LNPs were made using the T-tube formulation method at total flow rate of 20 mL/min and flow rate ratio of 3:1 aqueous: organic phases (v/v) with an initial lipid concentration of 10 μmol in 25% ethanol and 300 mM ammonium sulphate. Following formulation, particles were dialyzed against 300 mM ammonium sulphate using 12-14 kDa regenerated cellulose membranes (Spectrum Labs, Rancho Dominguez, 38 CA) overnight to remove residual EtOH. Prior to drug loading (see Section 4.3 Remote loading of doxorubicin into preformed liposomes), particles were dialyzed against Dulbecco's phosphate buffered saline (PBS) (pH 7.4) overnight using 12-14 kDa regenerated cellulose membranes. Cholesterol concentration of the particles was measured using the Wako Cholesterol E assay (Mountain View, CA) and used to determine the total lipid concentration.

Remote loading of Doxorubicin into preformed liposomes: Prepared liposomes with ammonium sulphate gradient (see previous section) were combined with doxorubicin dissolved in PBS to a final concentration of 3 mg/mL total lipid and a drug:lipid (molar) ratio of 0.1. Loading mixture was incubated at 65° C. for 30 min before being dialyzed against PBS overnight to remove any unencapsulated doxorubicin. Dialyzed particles were sterile filtered using a 0.2 μm syringe filter (Pall, Ville St. Laurent, QB).

Characterization of Dox-paLNP and Dox-red-paLNP: Particle size and polydispersity index (PDI) were determined through dynamic light scattering (DLS) using the Malvern Zetasizer NanoZS (Worcestershire, UK). Reported values correspond to number mean diameters. Cholesterol concentration of the Doxorubicin-LNP particles was determined using the Wako Cholesterol E assay (Mountain View, CA) and used to determine the total lipid concentration. The concentration of Doxorubicin in the loaded particles was measured using absorbance at 492 nm. Dox-Control LNP, dox-paLNP or dox-red-paLNP samples were collected prior to the incubation step in the loading procedure as well as post-loading and dialysis. The samples were mixed with 0.5% Triton X-100 in PBS at a dilution 1:20 dilution in a 96 well plate. After shaking briefly and incubating at room temperature for 5 minutes, absorbance values were measured at 492 nm. Encapsulation efficiency (percent encapsulation) was determined through comparison of the drug:lipid ratio of Dox-LNP pre-loading and post-loading and dialysis to remove unencapsulated doxorubicin (see Section 2.3 Remote loading of doxorubicin into preformed liposomes). Drug:lipid ratios were determined using the molar concentrations of doxorubicin and total lipid determined through A492 and Wako Cholesterol E assay, respectively.

Cryogenic transmission electron microscopy (Cryo-TEM) imaging of dox-control LNP and dox-paLNP: Control-LNP and paLNPs loaded with doxorubicin (0.1 drug:lipid, molar) were concentrated (Amicon Ultra-15 Centrifuge Filter Units, Millipore, Billerica, MA) to a total lipid concentration of ˜25 mg/mL prior to analysis. Some samples were subject to light-triggered drug release (see section 4.6) and compared to non-UV treated samples. Morphological liposome characteristics and doxorubicin loading were investigated by Cryo-TEM as described previously. In brief, liposome formulations were deposited onto glow-discharged copper grids and vitrified using a FEI Mark IV Vitrobot (FEI, Hillsboro, OR). Cryo-TEM imaging was performed using a 200 kV Glacios microscope equipped with a Falcon III camera at the UBC High Resolution Macromolecular Cryo-Electron Microscopy facility (Vancouver, BC).

Drug release assay of Dox-LNP incubated in PBS: Control-LNP and paLNP particles diluted to a final concentration of 3 mg/mL total lipid in PBS were irradiated with a UV-A light source (365 nm) for 5 minutes followed by storage in the dark at room temperature. At the 1 h time point, the incubated sample was passed down a size exclusion column to remove free drug. The drug:lipid ratio of purified LNPs was determined as detailed in the previous section and percent retention calculated relative to the t=0 h time point. Release experiments were repeated using pulsed irradiation at 365 nm over a 24 h period in the dark at room temperature, following which drug release was determined based on drug:lipid ratio as detailed in the previous section and percent retention calculated relative to the t=0 h time point.

Drug release assay of Dox-LNP incubated in DMEM media containing 10% FBS: Control-LNP and paLNP particles loaded with doxorubicin (0.1 drug:lipid ratio) at a final concentration of 3 mg/mL total lipid in DMEM media containing 10% FBS were irradiated with a UV-A light source (365 nm) for 15 min followed by pulsed irradiation at 365 nm over a 24 h period in the dark at room temperature. After 24 h, an aliquot was passed down a size exclusion column to remove free drug, followed by addition of a fixed ratio of isopropanol (IPA) to precipitate the proteins. The supernatant was analyzed to determine the drug: lipid ratio as detailed in the previous section and percent retention calculated relative to the t=0 h time point.

In vitro cell viability assay: Cell viability assay was performed using HuH7 cells—hepatocyte derived carcinoma cell line. Growth media was composed of DMEM with FBS (10%). Cells were plated in 96-well cell culture treated plates (Falcon/Corning Inc., Corning, NY) at a density of 12500 cells/well approximately 24 h prior to treatment. Either free doxorubicin, Control-LNP, paLNPs or red-paLNPs (0.1 drug:lipid ratio) in PBS were diluted as necessary with PBS and added to the appropriate volume of media to obtain final treatment concentrations of 0, 0.1, 1, 10 and 100 μM doxorubicin (free drug, Control-LNP or paLNP). Treated cells were subject to irradiation with UV-A light (365 nm) or deep-red light (660 nm) for 15 min following by pulsed irradiation (365 nm or 660 nm) incubated at 37° C. and 5% CO₂ for a total of 24 h. At the 24 h time point, cell viability was analyzed using an MTT assay (Abcam Inc.) comparing UV irradiated and non-UV irradiated cells.

Confocal imaging: Imaging was performed using HuH7 cells—hepatocyte derived carcinoma cell line. Growth media was composed of DMEM with FBS (10%). Cells were plated in confocal imaging plates at a density of 40,000 cells/well 24 h prior to treatment. Either free doxorubicin, Control-LNP or paLNPs (0.1 drug:lipid ratio) in PBS were diluted as necessary with PBS and added to the appropriate volume of media to obtain final treatment concentrations of 10 μM doxorubicin (free drug, Control-LNP or paLNP). Treated cells were subject to irradiation with UV-A light (365 nm) for 15 min following by pulsed irradiation (365 nm) incubated at 37° C. and 5% CO2 for a total of 24 hours. At the 24 h time point, cell membranes were stained with Cell Mask Deep Red Plasma Membrane Stain (1.0 mg/mL, Thermo Fisher Scientific). Live cell imaging comparing UV irradiated and non-UV irradiated cells was conducted using a Leica TCS SP8 laser scanning confocal microscope (Leica, Germany), equipped with a 60× oil-immersion objective (numerical aperture 1.40). Doxorubicin was excited at 488 nm argon laser and CellMask was excited with a 633 nm.

Zebrafish pharmacokinetic studies: Embryos from Tg(kdrl:eGFP) and Tg(AB/Tübingen) adult zebrafish (Danio rerio) were breaded at 28° C. in zebrafish culture media containing 30.4 ug/mL 1-phenyl-2-thiourea (PTU) and maintained according to Swiss animal welfare regulations. Embryos were embedded in 0.3% agarose containing tricaine and PTU and injected with a calibrated volume of 1 nL into Duct of Cuvier (48 hpf) and cardinal vein (72 hpf) using a micromanipulator (Wagner Instrumentenbau KG), a pneumatic Pico Pump PV830 (WPI) and a Leica S8APO microscope (Leica). A portion of injected fish was exposed, starting 0.16 hpi, to the corresponding pulsed light trigger at 28° C. for 24 h. The tail region of zebrafish embryos was imaged 2 and 24 h post-injection (hpi) using an Olympus FV3000 confocal laser scanning microscope equipped with a 30× UPIanSApo oil-immersion objective (NA 1.35). Quantitative image analysis was performed as previously described.

Doxorubicin release studies in zebrafish embryos: 48 hpf embryos from Tg(abc/tu) were injected with 2 nL of 3 mg/mL doxorubicin containing LNPs into the duct of Cuvier and incubated at 28° C. in PTU containing zebrafish culture media. 24 hpi, one set of zebrafish was irradiated with pulsed-light for additional 24 h. For quantitative analysis, confocal images of 5 fish were processed applying a defined threshold, followed by area calculation in Fiji (ImageJ) 2.1.0.

Although the present disclosure has been described with respect to one or more particular embodiments and/or examples, it will be understood that other embodiments and/or examples of the present disclosure may be made without departing from the scope of the present disclosure.

1. A photoswitchable glycerophospholipid comprising
a head group having a phosphate group,
one or two C₈-C₂₄ acyl groups, and
one or more photoswitchable groups,
wherein the photoswitchable group isomerizes upon exposure to electromagnetic radiation of the visible spectrum.

2. The photoswitchable glycerophospholipid according to claim 1, wherein the one or more photoswitchable groups have the following structure: 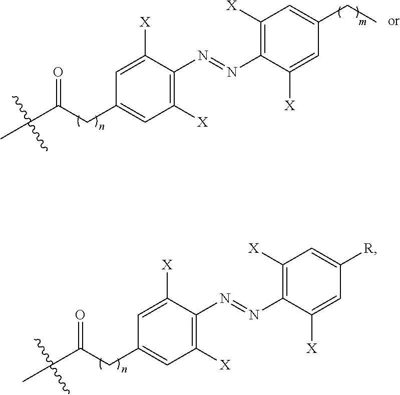
wherein each
X is individually chosen from —F, —Cl, and -OMe;
R is —H, —NO₂, amine groups, ether groups, —Br, or —I;
n is 0-12; and
m is 0-12.

3. The photoswitchable glycerophospholipid according claim 2, wherein the photoswitchable group has the following structure:

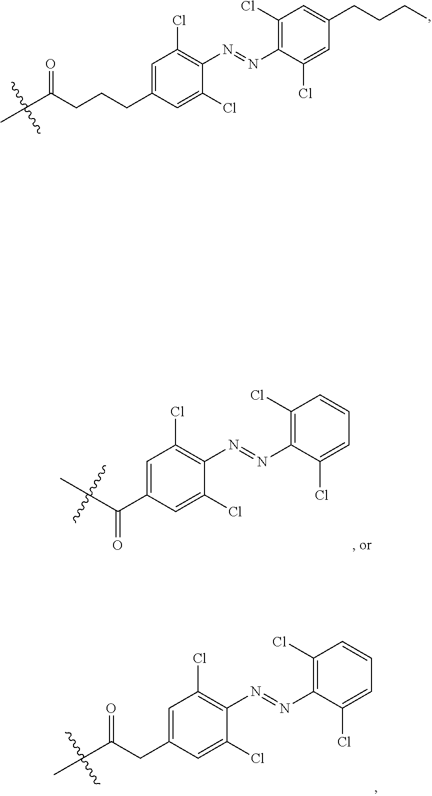

4. The photoswitchable glycerophospholipid according to claim 1, wherein the head group is chosen from an ethanolamine group, a choline group, a serine group, a glycerol group, a myo-inositol 4,5-bisphosphate group, and a phosphatidyl glycerol group.

5. The photoswitchable glycerophospholipid according to claim 1, wherein the photoswitchable glycerophospholipid has the following structure:

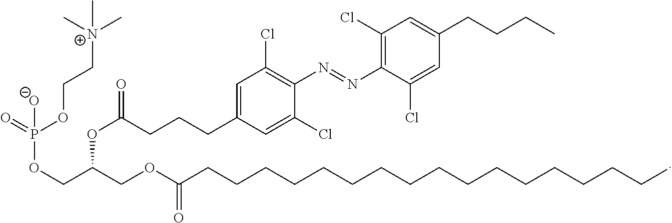

6. A nanovesicle comprising
one or more photoswitchable glycerophospholipid according to claim 1,
one or more glycerophospholipid that are not connected to a photoswitchable group, and
optionally, a sterol.

7. The nanovesicle according to claim 6, wherein the photoswitchable glycerophospholipid is present at a concentration of 0.1-80 mol %.

8. The nanovesicle according to claim 6, wherein the one or more glycerophospholipid is present at a concentration of 5-99.9 mol %.

9. The nanovesicle according to claim 6, wherein the sterol is present a concentration of 1 to 70 mol %,

10. The nanovesicle according to claim 9, wherein the sterol is chosen from cholesterol, sitosterol, stigmasterol, cholestanol, and combinations thereof.

11. The nanovesicle according to claim 6, further comprising one or more cargo molecules.

12. The nanovesicle according to claim 11, wherein the one or more cargo molecules are pharmaceutical drugs.

13. The nanovesicle according to claim 11, wherein the one or more cargo molecules are chosen from doxorubicin, nucleic acids, peptides, and combinations thereof.

14. The nanovesicle according to claim 6, wherein the nanovesicles have a longest linear dimension of 10-150 nm.

15. A composition comprising one or more nanovesicles according to claim 6 and one or more pharmaceutically acceptable carriers.

16. The composition according to claim 15, further comprising one or more cargo molecules.

17. The composition according to claim 16, wherein the one or more cargo molecules are pharmaceutical drugs.

18. The composition according to claim 16, wherein the one or more cargo molecules are chosen from doxorubicin, nucleic acids, peptides, and combinations thereof.

19. A method of delivering cargo molecules of one or more nanovesicles according to claim 11 to an individual comprising:
administering to the individual a composition comprising the nanovesicles, such that the nanovesicles are delivered to a target site;
exposing the target site of the individual to red spectral light,
wherein the one or more cargo molecules are released from the one or more nanovesicles and the cargo molecules are delivered to the target site of the individual.

20. The method according to claim 19, wherein the one or more cargo molecules are pharmaceutical drugs.

21. The method according to claim 20, wherein the one or more cargo molecules are chosen from doxorubicin, nucleic acids, peptides, and combinations thereof.

22. The method according to claim 19, wherein the red spectral light is produced by a scope comprising a light source.
